(19) United States
(12) Patent Application Publication
(10) Pub. No.: US 2011/0237000 A1
(43) Pub. Date: Sep. 29, 2011

(54) Method for detecting an analyte molecule

(75) Inventors: Ju Nie Tey, Singapore (SG); I Putu Mahendra Wijaya, Singapore (SG); Jun Wei, Singapore (SG); Isabel Rodriguez, Singapore (SG); Subodh Mhaisalkar, Singapore (SG)

(73) Assignees: Agency for Science, Technology and Research, Connexis (SG); Nanyang Technological University, Singapore (SG)

(21) Appl. No.: 13/065,051

(22) Filed: Mar. 11, 2011

Related U.S. Application Data
(60) Provisional application No. 61/313,028, filed on Mar. 11, 2010.

(51) Int. Cl. G01N 33/543; G01N 33/15; G01N 27/00; B82Y 35/00; B82Y 15/00

(52) U.S. Cl. 436/518; 422/82.02; 977/773; 977/742; 977/700; 977/904

(57) ABSTRACT

The invention relates to a method for detecting the presence or amount of an analyte, said method comprising (a) coupling the analyte to a carrier molecule, wherein the carrier molecule is larger in size, electrically charged and/or polar, to form an analyte:carrier molecule complex; (b) contacting the analyte:carrier molecule complex of (a) with an analyte-binding molecule coupled to a semiconducting nanostructure; and (c) determining the change in conductance upon binding of the analyte:carrier molecule complex to the analyte-binding molecule and correlating the determined change in conductance to the presence or amount of the analyte. Alternatively, the analyte:carrier molecule complex of (a) is immobilized on the nanostructure and the immobilized analyte:carrier molecule complex is contacted with the analyte-binding molecule.

### CROSS-REFERENCE TO RELATED APPLICATION

This application claims the benefit of priority of U.S. Provisional Application No. 61/313,028, filed 11 Mar. 2010, the contents of which being hereby incorporated by reference it its entirety for all purposes.

### TECHNICAL FIELD

The invention relates to a method of detecting the presence or amount of an analyte.

### BACKGROUND

Carbon nanotubes (CNTs) have attracted great attention in the field of sensing due to their exceptional charge transport characteristics which are confined to the nanotube surface and are able to detect molecular level changes in their immediate environment. Ever since the first demonstration of CNT sensing capability on gas molecules, numerous studies have reported on the interaction of CNTs with a variety of biological and bioactive species such as proteins, peptides, DNA, enzymes, and the ability to transduce this interaction into an effective sensor. By virtue of their small size, the sensitivity of the detecting or sensing elements such as nanotubes and nanowires, and the versatility to detect specific bindings of a vast group of analyte:analyte-binding molecules, nanostructure-based biosensors are rapidly gaining employability in real-time detection of the presence of biological molecules. For instance, single-walled carbon nanotubes (SWCNTs) have been employed in the detection of deoxyribonucleic acid (DNA) based on both electrochemical and transistor configuration and detection limits of the order of parts per trillion have been reported.

For the transduction of the sensing signal, diverse CNT sensor architectures have been employed. While popular electrochemistry approaches include CNT incorporated electrodes combined with cyclic voltammetry, amperometry, or impedance spectrometry, these devices normally require integration with electrochemical tags in order to translate surface binding phenomena into a readable signal. In contrast, field effect transistor (FET) based sensors allow analyte-receptor interaction to be directly translated into a readable electrical signal by monitoring conductance change in semiconducting CNTs (single, multiple, or networks) deposited across metal electrodes and functioning as the sensing interface. Selectivity of the sensing platform is accomplished by a specific recognition group (also called a receptor, ligand or probe) anchored to the CNTs that undergoes specific binding with the target analyte, an interaction that has much similarity with standard immunoassays. Compared with dry state measurements, liquid-gated field effect transistors (LGFET) are preferred for biosensing applications primarily because they are amenable to real-time detection at voltages less than 1 V and because the buffered liquid based environment is suitable for detection of biomolecules directly from serous fluids including blood serum and saliva. Liquid-gated devices are often based on nanotubes supported on a silicon substrate with a microfluidic channel defined in a poly dimethoxy siloxane (PDMS) film.

However, to date these applications have been limited to the detection of target analytes larger than 30 kDa such as proteins, antibodies and large antigens, or strongly charged molecules such as DNA, as only these types of analytes are capable of affecting the nanotube environment to an extent that a detectable signal change is generated.

Thus there exists the need for development of a simple and sensitive method that also allows detection of other types of analytes.

### SUMMARY

The present invention meets this need and provides for a sensitive method that allows the detection of analyte molecules by use of semiconducting nanostructures in a transistor- or resistor-based sensor.

In a first aspect, the present invention thus relates to a method for detecting the presence or amount of an analyte, said method comprising:

(a) coupling the analyte to a carrier molecule, wherein the carrier molecule is larger in size, electrically charged and/or polar, to form an analyte:carrier molecule complex;

(b) contacting the analyte:carrier molecule complex of (a) with an analyte-binding molecule coupled to a semiconducting nanostructure; and

(c) determining the change in conductance upon binding of the analyte:carrier molecule complex to the analyte-binding molecule and correlating the determined change in conductance to the presence or amount of the analyte.

In another aspect, the present invention relates to a method for detecting the presence or amount of an analyte, said method comprising:

(a) coupling the analyte to a carrier molecule, wherein the carrier molecule is larger in size, electrically charged and/or polar, to form an analyte:carrier molecule complex;

(b) immobilizing the analyte:carrier molecule complex of (a) on a semiconducting nanostructure;

(c) contacting the immobilized analyte:carrier molecule complex with an analyte-binding molecule; and

(d) determining the change in conductance upon binding of the analyte-binding molecule to the immobilized analyte:carrier molecule complex and correlating the determined change in conductance to the presence or amount of the analyte.

In a further aspect, the present invention relates to a fluidic sensor device for determining the presence of an analyte in a fluid sample, the sensor device comprising:
a substrate comprising a microchannel, wherein the microchannel comprises a detection area, and wherein the detection area is arranged to be contactable by the fluid sample flowing through the microchannel, wherein the detection area comprises a network of semiconducting nanostructures, wherein an analyte:carrier molecule complex or an analyte-binding molecule is coupled to the network of nanostructures; and
a first electrode and a second electrode, wherein the first electrode and the second electrode are electrically connected to the detection area.

### BRIEF DESCRIPTION OF THE DRAWINGS

In the drawings, like reference characters generally refer to the same parts throughout the different views. The drawings are not necessarily drawn to scale, emphasis instead generally being placed upon illustrating the principles of various embodiments. In the following description, various embodiments of the invention are described with reference to the following drawings.

FIG. 1 shows an outline of the method steps for detecting an analyte in one embodiment of the invention.

FIG. 2 shows a schematic laboratory set-up of the device for detecting analyte in one embodiment of the invention.

FIG. 3 shows a process flow for fabricating a biosensor used in the detecting methodologies in accordance with various embodiments of the invention. (a) A thin SWCNT network obtained by solution processing/filtration is stamped and transferred onto a PDMS substrate (top). A dense SWCNT network with defined source-drain pad and channel width (W) is stamped onto another PDMS substrate (bottom) with microfluidic channels defined on it by casting. The channel length (L) of the transistor is auto-defined by the width of the microchannel. (b) The transistor fabrication process is completed by sealing against both PDMS substrates. (c) Biosensing experiment is performed by pumping electrolyte solution into the microchannel of the biosensor and a reference electrode is inserted into the reservoir. The reservoir is also used for electrolyte refilling and sample solution injection.

FIG. 4 shows the Atomic Force Microscopy phase profile and its respective height analysis for (a) bare carbon nanotube network, (b) Morphine antibody attachment, and (c) 6-monoacetylmorphine-bovine serum albumin conjugate attachment onto the biosensor of FIG. 3, and (d) the respective X-ray Photoelectron Spectroscopy characterization of the covalent attachment through the carboxylic functional group via carbodiimide-activated amidation process.

FIG. 5 shows the effects of (a) pH and (b) ionic strength of electrolyte solutions on the conductance of the biosensor of FIG. 3.

FIG. 6 shows the selectivity study demonstrating the specificity of the carbon nanotube of the biosensor of FIG. 3 towards morphine antibody detection after overnight incubation with 6-monoacetylmorphine-bovine serum albumin conjugates and blocking with blocking agent tween-20.

FIG. 7 shows the effect of signal enhancement by gold nanoparticles injected into the bare carbon nanotube network of the biosensor of FIG. 3 at (a) pH 9.5, (b) pH 7.4, and (c) pH 4.5.

FIG. 8 shows a schematic illustration of three different assay detection schemes with (a) MAM, (b) Mor-Ab, and (c) Au labeled Mor-Ab (Au-Mor-Ab), respectively, in one embodiment of the invention.

FIG. 9 shows a comparison of the respective I_{DS}-V_{G} curves for the assay detection schemes of FIG. 8(a) and FIG. 8(b).

FIG. 10 shows (a) kinetic measurements and (b) concentration study of the detection scheme of FIG. 8(b), with concentrations varying from 10 fg ml⁻¹, 100 fg ml⁻¹, 1 pg ml⁻¹, 100 pg ml⁻¹, 1 ng ml⁻¹, 10 ng ml⁻¹, 100 ng ml⁻¹, 1 μg ml⁻¹, and 10 μg ml⁻¹, respectively.

FIG. 11 shows the (a) kinetic measurements and (b) concentration study of the detection scheme of FIG. 8(c), with concentrations varying from 1 fg ml⁻¹, 10 fg ml⁻¹, 100 fg ml⁻¹, 1 pg ml⁻¹, 100 pg ml⁻¹, 1 ng ml⁻¹, 10 ng ml⁻¹, 100 ng ml⁻¹, 1 μg ml⁻¹, and 10 μg ml⁻¹, respectively.

FIG. 12 shows the kinetic measurements of the three assay detection schemes of FIG. 8 upon injections of MAM, Mor-Ab, and Au-Mor-Ab, respectively.

FIG. 13 shows a schematic illustration of competitive assay detection scheme of MAM in one embodiment of the invention.

FIG. 14 shows the (a) kinetic measurements and (b) concentration study of the competitive detection scheme of FIG. 13, with concentrations varying from 1 μg ml⁻¹, 100 ng ml⁻¹, 10 ng ml⁻¹, 1 ng ml⁻¹, 100 pg ml⁻¹, 1 pg ml⁻¹, 100 fg ml⁻¹, 10 fg ml⁻¹, and 1 fg ml⁻¹, respectively.

FIG. 15 shows (a) a schematic illustration of direct assay detection scheme of 2,4-D, and (b) corresponding I_{DS}-V_{G} curves.

FIG. 16 shows (a) a schematic illustration of reverse assay detection scheme of anti-2,4-D, and (b) corresponding I_{DS}-V_{G} curves in another embodiment of the invention.

FIG. 17 shows (a) the drift characteristic of the LGFET in blank PBS solution, (b) kinetic measurements during the immunocomplex formation, (c) calibration curve resultant from the immunoreaction, and (d) study of specificity of the LGFET.

FIG. 18 shows (a) a schematic illustration of competitive assay detection scheme of 2,4-D, (b) corresponding kinetic measurements at different concentrations of free 2,4-D in PBS solution, and (c) calibration curve resultant from the immunoreaction in another embodiment of the invention.

FIG. 19 shows (a) kinetic measurements at different concentrations of free 2,4-D in soil sample, and (b) calibration curve resultant from the immunoreaction in another embodiment of the invention.

FIG. 20 shows a schematic illustration of direct assay detection scheme of (a) atrazine and (b) atrazine-BSA conjugate, (c) corresponding I_{DS}-V_{G} curve for direct atrazine detection scheme and (d) kinetic measurements of atrazine-BSA conjugate in another embodiment of the invention.

### DESCRIPTION

The following detailed description refers to the accompanying drawings that show, by way of illustration, specific details and embodiments in which the invention may be practised. These embodiments are described in sufficient detail to enable those skilled in the art to practise the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

The invention is based on the finding that the sensitivity of the detection of analyte molecules, which may be present in a sample in only trace amounts, can be significantly improved by coupling the analyte molecules to carrier molecules and detecting the binding event between the analyte:carrier molecule complex and analyte-binding molecules via semiconducting nanostructures, such as carbon semiconductor nanotubes or nanowires. By use of the present method, an analyte concentration as low as about 500 fM (i.e. 0.5 part-per-trillion) can be detected. The increased sensitivity of the detection method of the invention compared to other procedures known in the art can be attributed in part by the high sensitivity of the semiconducting nanostructures, for example carbon nanotubes or nanowires, to the slightest electrostatic disturbances in their surroundings. The present method thus aims to cause electrostatic disturbances in the surroundings of the carbon semiconductor nanotubes or nanowires by an analyte-binding event and correlating the detected change in the electric field to the presence or amount of the analyte. The present method could be implemented in an all-plastic, and therefore low cost fluid sensor device, demonstrating great potential for adoption for on-site analysis with high sensitivity and accuracy.

In a first aspect, the present invention relates to a method for detecting the presence or amount of an analyte, said method comprising:

(a) coupling the analyte to a carrier molecule, wherein the carrier molecule is larger in size, electrically charged and/or polar, to form an analyte:carrier molecule complex;

(b) contacting the analyte:carrier molecule complex of (a) with an analyte-binding molecule coupled to a semiconducting nanostructure; and

(c) determining the change in conductance upon binding of the analyte:carrier molecule complex to the analyte-binding molecule and correlating the determined change in conductance to the presence or amount of the analyte.

In a second aspect, the present invention relates to a method for detecting the presence or amount of an analyte, said method comprising:

(a) coupling the analyte to a carrier molecule, wherein the carrier molecule is larger in size, electrically charged and/or polar, to form an analyte:carrier molecule complex;

(b) immobilizing the analyte:carrier molecule complex of (a) on a semiconducting nanostructure;

(c) contacting the immobilized analyte:carrier molecule complex with an analyte-binding molecule; and

(d) determining the change in conductance upon binding of the analyte-binding molecule to the immobilized analyte:carrier molecule complex and correlating the determined change in conductance to the presence or amount of the analyte.

In a further aspect, the invention is particular useful as a microfluidic-based fluidic sensor device for determining the presence of an analyte in a fluid sample. The sensor device comprises:
a substrate comprising a microchannel, wherein the microchannel comprises a detection area, and wherein the detection area is arranged to be contactable by the fluid sample flowing through the microchannel, wherein the detection area comprises a network of semiconducting nanostructures, wherein an analyte:carrier molecule complex or an analyte-binding molecule is coupled to the network of nanostructures; and
a first electrode and a second electrode, wherein the first electrode and the second electrode are electrically connected to the detection area.

The terms “analyte”, “target compound”, “target molecule” or “target” as interchangeably used herein, refer to any substance that can be detected in an assay by binding to a binding molecule, and which, in one embodiment, may be present in a sample. Therefore, the analyte can be, without limitation, any substance for which there exists a naturally occurring antibody or for which an antibody can be prepared. The analyte may, for example, be a small organic compound such as a drug, toxin, herbicide, and metabolites thereof, dye, or other small molecule present in the sample. Additionally, however, the analyte may also be an antigen, a protein, a polypeptide, a nucleic acid, a hapten, an immunological hapten, a carbohydrate, a cell or any other of a wide variety of biological or non-biological molecules, complexes or combinations thereof. The analyte may be a protein, peptide, carbohydrate or lipid derived from a biological source such as bacterial, fungal, viral, plant or animal samples. The analyte may be a small molecule, such as a molecule with a molecular weight of below 1 kDa or of below 500 Da. Alternatively or additionally, the analyte may have no net charge and/or no dipole moment or a net charge and/or dipole moment that is too small to allow detection of the analyte in the absence of a suitable carrier.

The term “hapten” as used herein, refers to a small proteinaceous or non-protein antigenic determinant which is capable of being recognized by an antibody. Typically, haptens do not elicit antibody formation in an animal unless part of a larger molecule. For example, small peptide haptens are frequently coupled to a carrier protein such as keyhole limpet hemocyanin in order to generate an anti-hapten antibody response.

“Antigens” are macromolecules capable of generating an antibody response in an animal and being recognized by the resulting antibody. Both antigens and haptens comprise at least one antigenic determinant or “epitope”, which is the region of the antigen or hapten which binds to the antibody. Typically, the epitope on a hapten is the entire molecule.

The term “sample”, as used herein, refers to an aliquot of material, frequently but not necessarily always the case biological matrices, an aqueous solution or an aqueous suspension derived from biological material. Samples to be assayed for the presence of an analyte by the methods of the present invention include, for example, contaminated (suspected or otherwise) water or other fluids, soil, cells, tissues, homogenates, lysates, extracts, and purified or partially purified proteins and other biological molecules and mixtures thereof.

Non-limiting examples of samples include human and animal body fluids such as whole blood, serum, plasma, cerebrospinal fluid, sputum, bronchial washing, bronchial aspirates, urine, semen, lymph fluids and various external secretions of the respiratory, intestinal and genitourinary tracts, tears, saliva, milk, white blood cells, myelomas and the like; biological fluids such as cell culture supernatants; tissue specimens which may or may not be fixed; and cell specimens which may or may not be fixed. The samples to be tested will vary based on the assay format and the nature of the tissues, cells, extracts or other materials, especially biological materials, to be assayed. Methods for preparing protein extracts from cells or samples are well known in the art and can be readily adapted in order to obtain a sample that is compatible with the methods of the invention. Detection in a body fluid can also be in vivo, i.e. without first collecting a sample.

“Peptide” generally refers to a short chain of amino acids linked by peptide bonds. Typically peptides comprise amino acid chains of about 2-100, more typically about 4-50, and most commonly about 6-20 amino acids. “Polypeptide” generally refers to individual straight or branched chain sequences of amino acids that are typically longer than peptides. “Polypeptides” usually comprise at least about 20 to 1000 amino acids in length, more typically at least about 100 to 600 amino acids, and frequently at least about 200 to about 500 amino acids. Included are homo-polymers of one specific amino acid, such as for example, poly-lysine. “Proteins” include single polypeptides as well as complexes of multiple polypeptide chains, which may be the same or different.

Multiple chains in a protein may be characterized by secondary, tertiary and quaternary structure as well as the primary amino acid sequence structure, may be held together, for example, by disulfide bonds, and may include post-synthetic modifications such as, without limitation, glycosylation, phosphorylation, truncations or other processing.

Antibodies such as IgG proteins, for example, are typically comprised of four polypeptide chains (i.e., two heavy and two light chains) that are held together by disulfide bonds. Furthermore, proteins may include additional components such associated metals (e.g., iron, copper and sulfur), or other moieties. The definitions of peptides, polypeptides and proteins includes, without limitation, biologically active and inactive forms; denatured and native forms; as well as variant, modified, truncated, hybrid, and chimeric forms thereof.

The term “analyte-binding molecule” as used herein refers to any molecule capable of binding to an analyte of choice so as to form a complex consisting of the analyte-binding molecule and the analyte. Preferably, this binding is specific so that a specific complex between analyte and analyte binding molecule is formed. The analyte-binding molecule may be selected from the group consisting of an antibody, antibody fragment, antibody variant, antibody-like molecule, binding protein and receptor protein and domains thereof.

“Specifically binding” and “specific binding” as used herein mean that the analyte-binding molecule binds to the target analyte based on recognition of a binding region or epitope on the analyte molecule. The analyte-binding molecule preferably recognizes and binds to the analyte molecule with a higher binding affinity than it binds to other compounds in the sample. In various embodiments of the invention, “specifically binding” may mean that an antibody or other biological molecule, binds to an analyte molecule with at least about a 10⁶-fold greater affinity, preferably at least about a 10⁷-fold greater affinity, more preferably at least about a 10⁸-fold greater affinity, and most preferably at least about a 10⁹-fold greater affinity than it binds molecules unrelated to the analyte molecule. Typically, specific binding refers to affinities in the range of about 10⁶-fold to about 10⁹-fold greater than non-specific binding. In some embodiments, specific binding may be characterized by affinities greater than 10⁹-fold over non-specific binding. The binding affinity may be determined by any suitable method. Such methods are known in the art and include, without limitation, surface plasmon resonance and isothermal titration calorimetry. In a specific embodiment, the analyte-binding molecule uniquely recognizes and binds to the analyte.

The analyte-binding molecule may be a proteinaceous molecule, such as an antibody, for example a monoclonal or polyclonal antibody, which immunologically binds to the analyte at a specific determinant or epitope. The term “antibody” is used in the broadest sense and specifically covers monoclonal antibodies as well as antibody variants or fragments (e.g., Fab, F(ab′)₂, scFv, Fv diabodies and linear antibodies), so long as they exhibit the desired binding activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. The monoclonal antibodies can include “chimeric” antibodies and humanized antibodies. A “chimeric” antibody is a molecule in which different portions are derived from different animal species, such as those having a variable region derived from a murine mAb and a human immunoglobulin constant region.

Monoclonal antibodies may be obtained by any technique that provides for the production of antibody molecules by continuous cell lines in culture. These include, but are not limited to the hybridoma technique of Koehler and Milstein (U.S. Pat. No. 4,376,110), the human B-cell hybridoma technique, and the EBV-hybridoma technique. Such antibodies may be of any immunoglobulin class including IgG, IgM, IgE, IgA, IgD and any subclass thereof. The hybridoma producing the mAb may be cultivated in vitro or in vivo. Production of high titres of mAbs in vivo makes this a very effective method of production.

“Polyclonal antibodies” are heterogeneous populations of antibody molecules derived from the sera of animals immunized with an antigen, or an antigenic functional derivative thereof. For the production of polyclonal antibodies, host animals such as rabbits, mice and goats, may be immunized by injection with an antigen or hapten-carrier conjugate optionally supplemented with adjuvants.

Alternatively, techniques described for the production of single chain antibodies (U.S. Pat. No. 4,946,778) can be used to produce suitable single chain antibodies. Single chain antibodies are typically formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed to allow rapid and easy identification of monoclonal Fab fragments with the desired specificity.

The analyte-binding molecule may also be any other proteinaceous scaffold that has been adapted or mutated to bind a given ligand with sufficient binding affinity. Examples of useful scaffolds include those scaffolds described in US patent application 2005/0089932 or U.S. Pat. No. 6,682,736. Another example of suitable scaffolds are members of the lipocalin protein family as described in the international patent applications WO 99/16873, WO 00/75308, WO 03/029471, WO 03/029462, WO 03/029463, WO 2005/019254, WO 2005/019255 or WO 2005/019256, for instance.

In accordance with the above, scaffolds besides members of the lipocalin family include, but are not limited to, a EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or a an immunoglobulin-like domain (for example, domain antibodies or camel heavy chain antibodies), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, Kappabodies, Minibodies, Janusins, a nanobody, a adnectin, a tetranectin, a microbody, an affilin, an affibody or an ankyrin, a crystallin, a knottin, ubiquitin, a zinc-finger protein, an ankyrin or ankyrin repeat protein or a leucine-rich repeat protein, an avimer; as well as multivalent avimer proteins evolved by exon shuffling of a family of human receptor domains.

As mentioned above, in certain embodiments of the invention the analyte-binding molecule may be a mutein of the member of the lipocalin protein family. In some of these embodiments, the open end of the β-barrel structure of the lipocalin fold (which encompasses the natural ligand binding site of the lipocalin family) is used to form the target analyte binding site. Members of the lipocalin family of proteins include, but are not limited to the bilin binding protein of Pieris brassicae (SWISS-PROT Data Bank Accession Number P09464), human tear lipocalin (SWISS-PROT Data Bank Accession Number M90424), human apolipoprotein D (SWISS-PROT Data Bank Accession Number P05090), the retinol binding protein (RBP) (for example of human or porcine origin, SWISS-PROT Data Bank Accession Number of the human RBP: P02753, SWISS-PROT Data Bank Accession Number of the porcine RBP P27485), human neutrophil gelatinase-associated lipocalin (hNGAL, SWISS-PROT Data. Bank Accession Number P80188), rat α₂-microglobulin-related protein (A2m, (SWISS-PROT Data Bank Accession Number P31052), and mouse 24p3/uterocalin (24p3, (SWISS-PROT Data Bank Accession Number P11672), Von Ebners gland protein 2 of Rattus norvegicus (VEG protein 2; SWISS-PROT Data Bank Accession Number P41244), Von Ebners gland protein 2 of Sus scrofra (pig) (LCN1; SWISS-PROT Data Bank Accession Number P53715), the Major allergen Can fl precursor of dog (ALL 1, SWISS-PROT Data Bank Accession Number O18873), and insecticyanin A or insecticyanin B of the tobacco hawkmoth Manducta sexta (SWISS-PROT Data Bank Accession Number P00305 and Q00630, respectively).

The analyte-binding molecule may also be a binding protein, receptor or extracellular domain (ECD) thereof capable of forming a binding complex with a ligand, typically a polypeptide or glycopeptide ligand.

Those skilled in the art will recognize that the non-limiting examples given above describing various forms of antibodies as analyte-binding molecules can also be extended to other proteinaceous receptors such as recombinant, chimeric, hybrid, truncated etc., forms of non-antibody receptors.

The analyte-binding molecule can also be a non-proteinaceous receptor, such as for example a nucleic acid based molecule, such as an Aptamer or Spiegelmer (Aptamer made of L-ribonucleotides).

The terms “contacting” or “incubating” as used interchangeably herein refer generally to providing access of one component, reagent, analyte or sample to another. For example, contacting can involve mixing a solution comprising an analyte binding protein or conjugate thereof with a sample. The solution comprising one component, reagent, analyte or sample may also comprise another component or reagent, such as dimethyl sulfoxide (DMSO) or a detergent, which facilitates mixing, interaction, uptake, or other physical or chemical phenomenon advantageous to the contact between components, reagents, analytes and/or samples.

The term “detecting” as used herein refers to a method of verifying the presence of a given molecule. The detection may also be quantitative, i.e. include correlating the detected signal with the amount of analyte. The detection includes in vitro as well as in vivo detection.

The term “conjugate” and its associated term “conjugated” as used herein refers to two or more molecules which have been linked or coupled together. The linkage or coupling to each other may be covalent or non-covalent, but preferably is covalent. In one embodiment of such a conjugate, the analyte is covalently coupled to the carrier molecule to form an analyte:carrier molecule complex. In another embodiment, the analyte:carrier molecule complex is covalently coupled or immobilized on the semiconducting nanostructure, for example a carbon semiconductor nanotube or nanowire. This coupling may be via the carrier molecule. In yet another embodiment, the analyte-binding molecule is covalently coupled to the semiconducting nanostructure, for example a carbon semiconductor nanotube or nanowire. In a further embodiment, the carrier molecule is conjugated to a signal enhancer, such as a metal nanoparticle. In yet another embodiment, the analyte-binding molecule is conjugated to a signal enhancer, such as a metal nanoparticle.

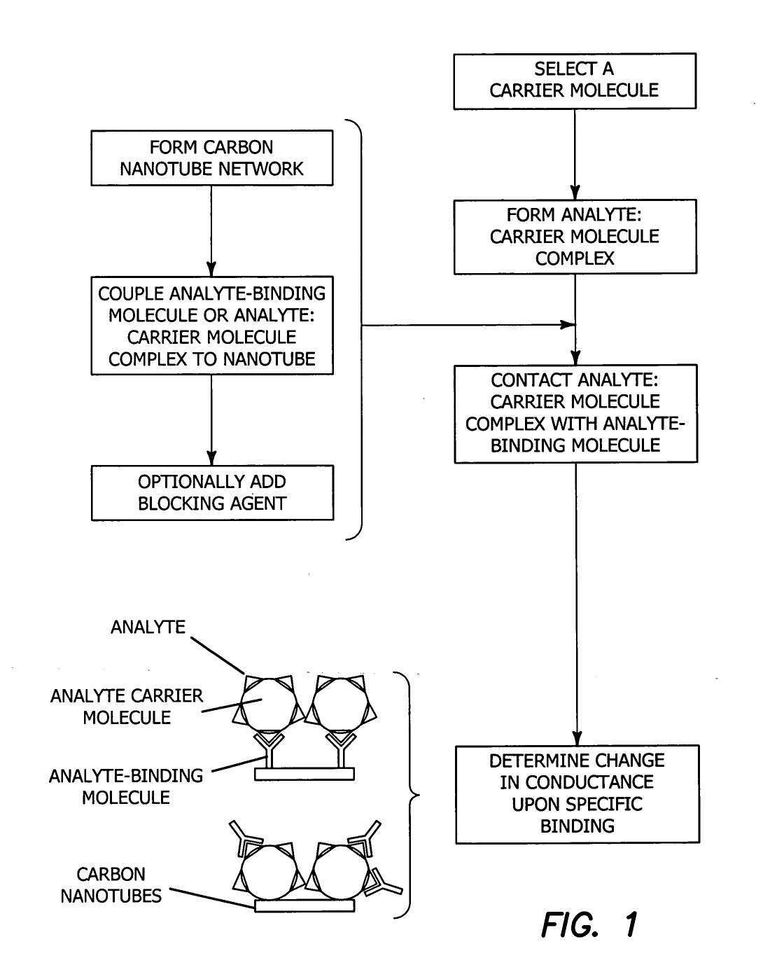

An exemplary embodiment of the invention is illustrated in FIG. 1, which shows that a selected carrier molecule is coupled to an analyte to form an analyte:carrier molecule complex. The analyte:carrier molecule complex is then contacted with an analyte-binding molecule in a detection area. The detection area is comprised in a microchannel of a substrate of a fluidic sensor device, for example. The detection area may comprise a network of semiconducting nanostructures. In various embodiments, the semiconducting nanostructures may comprise nanotubes, nanowires, nanopillars, nanorods, nanospheres, or a mixture thereof. In various embodiments, the semiconducting nanostructure may comprise or consist of carbon semiconductor nanotubes or nanowires. Although in the following paragraphs, the invention is disclosed with relation to carbon semiconductor nanotubes or nanowires forming the semiconducting nanostructure, it is to be understood and appreciated that the invention is not limited to such embodiments, but that the carbon semiconductor nanotubes or nanowires in any of the below embodiments can be replaced by other suitable semiconducting nanostructures. The detection area may be a part of a transistor-based or a resistor-based sensor device. For example, the sensor device may be a transistor such as a field effect transistor. In various embodiments, the sensor device may be a liquid-gated field effect transistor. Upon binding of the analyte-carrier molecule complex to the analyte-binding molecule, a signal is detected and determined. The signal may be, for example, a change in conductance detected by electrodes connected to the detection area. The determined change in conductance is then correlated to the presence or amount of the analyte in a sample.

Prior to contacting the analyte:carrier molecule complex with the analyte-binding molecule in the detection area, the sensing platform of the detecting transistor is first prepared, in which carbon semiconductor nanotubes or nanowires are randomly arranged on the transistor to form a network of interconnecting nanotubes or nanowires to conduct charges upon activation. The network of nanotubes or nanowires may be formed by vacuum filtration method, for example. The network of nanotubes or nanowires may comprise a network of nanotubes or nanowires in the form of a flexible, laminated network. The nanotubes or nanowires may be deposited across the electrodes connected to the detection area.

In one embodiment, analyte-binding molecules, for example antibody molecules, are then immobilized or coupled to the nanotubes or nanowires network through covalent or non-covalent binding. Such protocols where the analyte-binding molecule is initially coupled to the nanotubes or nanowires network prior to contacting with the analyte:carrier molecule complex are hereinafter termed direct detection assays.

Alternatively, the analyte:carrier molecule complex may be immobilized or coupled to the carbon nanotubes or nanowires network according to another embodiment. Such assays, where the analyte:carrier molecule complex is initially coupled to the nanotubes or nanowires network prior to contacting with the analyte-binding molecules, are hereinafter termed reverse detection assays. In one embodiment, a defined amount of the analyte-binding molecules is contacted with an unknown amount of analyte, for example a sample suspected of containing the analyte, prior to contacting the mixture of analyte-binding molecules and analyte with the immobilized analyte:carrier molecule complex. In such an embodiment, only the free analyte-binding molecules, i.e. those not bound to the analyte, are available for binding to the immobilized analyte:carrier molecule complex. The amount of free analyte-binding molecules depends on the amount of analyte in the sample. Such embodiments are hereinafter termed competitive detection assays. In such competitive detection assays, the change in conductance is inversely proportional to the amount of the analyte present in a sample. A higher amount of analytes present in the sample would result in a lesser amount of free analyte-binding molecules available for binding to the immobilized complex since more analyte-binding molecules would have bound to the analytes, and this would therefore translate to a smaller conductance change detected.

After immobilizing either the analyte-binding molecule or the analyte:carrier molecule complex to the nanotubes or nanowires network, a blocking agent may optionally be incubated on the nanotubes or nanowires network to block out remaining available carbon nanotubes or nanowires reactive sites to prevent non-specific binding of subsequently introduced analyte:carrier molecule complex or analyte-binding molecule to the nanotubes or nanowires network.

The methods as described above are particularly useful for detecting analyte molecules which may be neutral, i.e. do not bear a net charge, have no dipole moment, or any other analyte molecule that, in the absence of a suitable carrier, would only induce a small electrical signal. The small signal may be attributed to, for example, the small molecule size, charge neutrality, absence of dipoles, or the small net charge/dipole moment of the analyte molecules. In one embodiment, “small” with relation to analyte, means that the analyte has a molecular mass or size of below 1 kDa. In another embodiment, the analyte has a molecular mass of below 500 Da. In certain embodiments of the invention, the carrier molecule has a size that is about 10 times larger than that of the analyte. In one embodiment, the carrier molecule has a size of above 1 kDa or above 5 kDa. In certain embodiments of the invention, the analyte-binding molecule has a size that is about 10 times larger than that of the analyte. In one embodiment, the analyte-binding molecule has a size that is about 100 times larger than that of the analyte. In one embodiment, the analyte-binding molecule has a size of above 5 kDa or above 50 kDa.

In one embodiment, the analyte may be a drug molecule such as morphine, imatinib, or paracetamol. In various embodiments, the analyte may be neurotransmitters such as dopamine, epinephrine, acetylcholine, glutamate, or glycine.

In another embodiment, the analyte may be a pesticide such as atrazine or 2,4-dichlorophenoxyacetic acid. In various embodiments, the analyte may be aminopyralid, clopyralid, triclopyr, chlorpropham, or azinphosmethyl.

In one embodiment, the analyte is a neutral charge analyte, i.e. bears no net charge either by being uncharged or by bearing the same number of positive and negative charges.

Embodiments of the invention may be applied to the testing of water quality, or other chemical substances for purity, or for the presence of specific types of molecules. For example, using a suitable antibody, the invention can be used to detect biomarkers. Biomarkers are used in various applications, such as medical and diagnostic applications, for example the diagnosis of cancers (p53, Prostate Specific Antigen (PSA)).

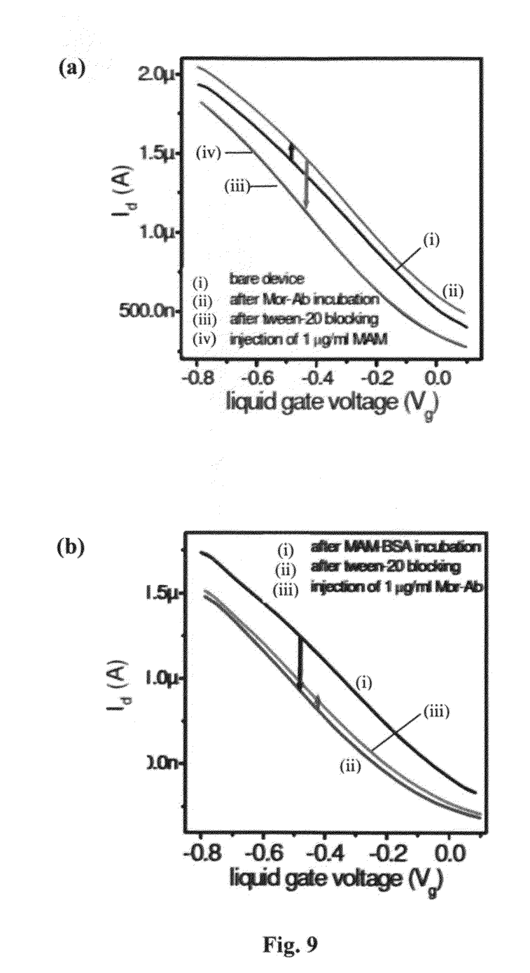

As mentioned earlier, the analyte-binding molecules may be any molecules that bind specifically to the analytes of interest, which binding event translates to a signal useful for determining the presence or amount of the analytes. In this context, a signal is considered useful when the signal is detectable and, when detected, it is distinct from the signal detected without having the binding event taking place. In one example illustrated in following paragraphs under the “Examples” section, when analyte-binding molecules (antibodies) were immobilized on the detection area (which is a microfluidic channel of a liquid-gated field effect transistor comprising a network of carbon semiconductor nanotubes), the analyte-binding molecules-functionalization of the detection area showed promising change in drain current (I_{DS}) (FIG. 9(a)). However, no detectable signal was observed upon injection of the analytes (morphine). In other words, even before introducing the analytes into the detection area, an electrial signal may be already detected when analyte-binding molecules (or in certain cases, analyte:carrier molecule complexes) are immobilized on the nanotubes or nanowires network in the detection area due to the inherent charged polarity of the analyte-binding molecules (or analyte:carrier molecule complexes). This initial signal may be considered to be the baseline. Upon analyte injection, changes in conductance with respect to this baseline are considered to be the detection signal. The detectable signal of interest is to be distinguished from this initial immobilized analyte-binding molecule-induced (or analyte:carrier molecule complex-induced) baseline signal.

In one embodiment, the analyte-binding molecule may be an antibody. The antibody may be raised, prepared, and stored for usage for a given known antigen to be detected.

The carrier molecule may be any molecule that carries or couples to the analyte, without adversely influencing the ability of the coupled analyte to subsequently bind with the analyte-binding molecule. The coupling of the analyte to the carrier molecule may be a covalent coupling, for example. Alternatively, the coupling may be non-covalent, for example by hydrogen bonding, van der Waals forces, or electrostatic interactions or a combination thereof Compared to the analyte, the carrier molecule is larger in size, electrically charged and/or polar. Some carrier molecules may not have net electric charge, but instead have a polarity (also known as dipole moment). In such a case, the sensor device detects the dipole moment instead. When dipole moment is the origin of the signal, the orientation of molecule plays a role in deciding whether the conductance change is increasing (indicating negative end of the dipole moment) or decreasing (indicating positive end of the dipole moment). Thus, in the case of polar carrier molecules, the conductance change also give information about the orientation of the polar carrier molecules. The carrier molecule may have a molecular mass of above 1 kDa, for example above 5 kDa.

In one embodiment, the carrier molecule may be a macromolecule such as a protein, which is but one example of a carrier molecule. The protein may be an albumin. In one embodiment, the carrier molecule is bovine serum albumin.

Advantageously, the carrier molecule, when coupled to the analyte, forms an analyte:carrier molecule complex which imposes a detectable signal disturbance to the network of carbon semiconductor nanotubes or nanowires upon the binding event of the analyte and the analyte-binding molecule. In one embodiment, this signal disturbance constitutes the detectable signal. In another embodiment, this signal disturbance imposed by the analyte:carrier molecule complex together with signal amplification imposed by a signal enhancer constitute the detectable signal. The signal enhancer may comprise, for example, a metal nanoparticle conjugated to the carrier molecule or the analyte-binding molecule.

In order to impose a signal disturbance to the nanotubes or nanowires network such that a detectable signal is observed and determined, the analyte:carrier molecule complex to be formed preferably has a size of above 1 kDa. In one embodiment, the analyte:carrier molecule complex has a size of above 5 kDa. The carrier molecule may thus be selected to fulfill this size requirement. It is to be understood and appreciated that the coupling of the analyte to the carrier molecule is in itself a form of signal amplification strategy because the signal from the small analyte has been magnified significantly after coupling with the larger carrier molecule. This signal amplification may be further enhanced by addition of a signal enhancer, as will be discussed in subsequent paragraphs.

The analyte:carrier molecule complex or analyte-binding molecule, or both, may optionally have a conjugated signal enhancer. In one embodiment, the signal enhancer comprises a nanoparticle, for example a metal nanoparticle. “Nanoparticle” as used herein relates to a particle sized between 1 and 100 nanometers. In one embodiment, the metal nanoparticle comprises or consists of a metal selected from the group consisting of copper, gold, silver, and platinum.

In another embodiment, the signal enhancer comprises a semiconducting silicon particle or a silica particle or a quantum dot. The signal enhancer may also comprise an organic molecule such as a dendrimer.

In yet another embodiment, the signal enhancer comprises a nanostructured material, for example a carbon-based nanomaterial such as a carbon nanotube, a nanowire, a modified carbon nanotube, a modified nanowire, or a fullerene. In one embodiment, analyte-binding molecules may be conjugated to a carbon nanotube signal enhancer in a competitive assay detection scheme. Due to the length of the carbon nanotube, a plurality of the analyte-binding molecules may be bound onto the nanotube surface. When the analyte-binding molecules coupled to the nanotube bind to the analyte coupled to the semiconducting nanostructures in the detection area, a high conductance change can be detected. The nanostructured material signal enhancer may further comprise a complex structure consisting of biomolecules bound to the nanostructures, the biomolecules may include the carrier molecule, the analyte:carrier molecule complex, the analyte-binding molecule, enzyme, or the like.

The choice of the signal enhancer is dependent on the sensing system. For example, to amplify the signal of a positively charged analyte, or positively charged analyte:carrier molecule complex, where the electrical current decreases upon the binding of the analyte with the analyte-binding molecule, a positively charged signal enhancer is used. To amplify the signal of a negatively charged analyte, or negatively charged analyte:carrier molecule complex, where the electrical current increases upon the binding of the analyte with the analyte-binding molecule, a negatively charged signal enhancer is used.

Advantageously, the carrier molecule is coupled to two or more analyte molecules to form an analyte:carrier molecule complex comprising two or more analyte molecules. This increases the density of the analyte molecules in the detection area, and hence allows a higher probability for binding with the analyte-binding molecules.

Furthermore, in certain embodiments the analyte:carrier molecule complex is immobilized or coupled to the network instead (for example, the reverse or competitive assay detection scheme). In such embodiments, the carrier molecule may be selected to have high affinity to the nanotubes or nanowires network.

Alternatively, a network of pristine carbon semiconductor nanotubes or nanowires may be treated to form functionalized nanotubes or nanowires network. In one embodiment, the nanotubes or nanowires are acid-treated to form carboxylated nanotubes or nanowires to facilitate the formation of a coupling bond with the analyte:carrier molecule complex or analyte-binding molecule, wherein the coupling is sufficiently strong to withstand multiple rinsing and washing thereby forming a stable and robust nanotubes or nanowires network. The presence of the carboxylic group may facilitate interaction between the coupled analyte:carrier molecule complex or analyte-binding molecule and the nanotubes or nanowires. The carboxylic group can also form various derivatives in different conditions to provide desirable functional groups such as arenes, alkyl halide, alcohol, ether, amine, aldehyde, ester, thiol, or the like. The presence of one or more of these functional groups may provide a desirable functionality to the nanotubes or nanowires depending on the application. However, the presence of carboxylic group is not necessary in some embodiments.

In one embodiment, the carbon semiconductor nanotube or nanowire is selected from the group consisting of a single nanotube or nanowire, multiple nanotubes or nanowires, or a network of nanotubes or nanowires.

In one embodiment, the carbon semiconductor nanotube or nanowire is deposited across metal electrodes connected to the detection area.

In one embodiment, the carbon semiconductor nanotube or nanowire comprises a network of nanotubes or nanowires in the form of, a flexible, laminated network. The nanotube or nanowire network may be placed in a microfluidic channel. The nanotube or nanowire network in the microfluidic channel forms part of a liquid-gated field effect transistor, for example.

In one embodiment, the carbon nanotube comprises a single-wall carbon nanotube (SWCNT). In an alternative embodiment, the carbon nanotube comprises a multi-walled carbon nanotube (MWCNT). Preferably, the carbon nanotube comprises SWCNT. SWCNTs comprise single layers of graphene sheets wrapped into a nano-sized tubular structure, and consequently charge-carriers are confined purely to the surface of the nanotubes. The conductivity of nanotubes is thus very sensitive to the slightest electrostatic disturbances in its surroundings. Biomolecule/SWCNT interactions lead to electrostatic potential disturbance and modulates the nanotube conductance, hence provide a facile transduction mechanism discernible with a sensor.

Preferably, the detection area forms part of the microfluidic channel of an integrated fluidic sensor device for real-time detection of analytes in small amount of liquid media. In one embodiment, the detection area forms part of a transistor channel of a field-effect transistor (FET). In one embodiment, the nanotube network comprises a thin film of SWCNTs formed on the transistor channel. FET-based sensors allow analyte:analyte-binding molecule interaction to be directly translated into readable electrical signal by monitoring conductance change in semiconducting carbon nanotubes deposited across electrodes and functioning as the sensing interface. Selectivity of the sensing platform is accomplished by specific recognition group anchored to the nanotubes that undergoes specific binding with the analyte, an interaction that has much similarity with standard immunoassays. The FET thus-formed is a liquid-gated FET (LGFET) since the testing electrolyte is a liquid medium placed in the transistor microfluidic channel. Compared with dry-state measurements, LGFETs are preferred for biosensing applications primarily because they are amenable to real-time detection at voltages less than 1 V. Further, the buffered liquid-based environment is suitable for detection of biomolecules directly from serous fluids including blood serum and saliva. Advantageously, the LGFET is low cost and the instrumentation required for sensing is made field-deployable. Preferably, the LGFET is made entirely of plastic, such as poly(dimethyl siloxane) (PDMS), poly(methyl methacrylate) (PMMA), polycarbonate, or polyepoxide (epoxy), which are but few examples of plastic suitable for fabricating the LGFET.

A pair of electrodes (source and drain electrodes) is deposited across the transistor microfluidic channel comprising the nanotubes or nanowires network. The electrodes may be formed of a conducting nanostructure, a metal, or other suitable conducting materials, and may be deposited across the transistor microfluidic channel by transfer printing, or any other suitable techniques. The electrodes may be further connected to an ammeter and computer for reading and outputting the detected signal.

Suitable nanostructures for the electrodes include nanotubes, nanowires, nanorods, and nanopillars. In one embodiment, the nanowires may be formed of silicon, germanium, zinc oxide, copper(II)oxide, titanium oxide, or tin oxide, which are but few examples of suitable nanowires.

Suitable metals for the electrodes include, but are not limited to, gold, silver, platinum, chromium, and combination therein.

Other suitable conducting materials for the electrodes may include Ag/AgCl electrodes.

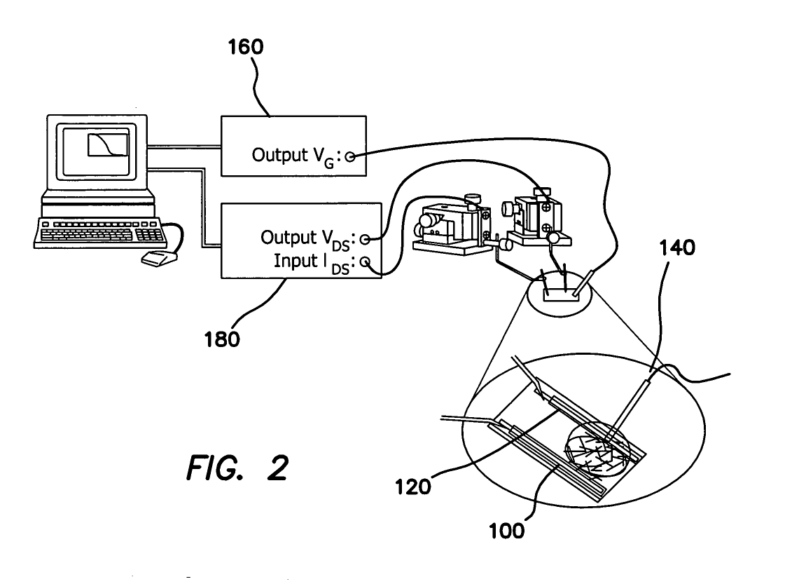

An exemplary embodiment of the biosensor set-up and measurement is illustrated in FIG. 2. A test liquid electrolyte is placed in a transistor channel 200 comprising a network of carbon nanotubes. A gate electrode 140 is in contact with the liquid electrolyte. A gate voltage is supplied from a function generator 160 to the liquid electrolyte (V_{G}). The gate voltage may be monitored using a suitable voltmeter such as an oscilloscope. Conveniently, the function generator 160 and the voltage monitor may be integrated.

A source electrode 100 and a drain electrode 120 are deposited across the transistor channel. A source-drain voltage (V_{DS}) is supplied from a suitable signal generator. The source-drain electrical current (I_{DS}) is measured using a suitable ammeter. The signal generator and the ammeter may be integrated and provided in ammeter-voltage source device 180. As the source-drain current is typically in the pico-ampere to micro-ampere range, a pico-ammeter with voltage source function may be used.

The function generator 160 and the ammeter-voltage source device 180 may be in communication with a computer, which includes a processor and processor readable storage medium. The storage medium may store a processor executable programme codes that, when executed by the processor, adapt the computer to control the operation of one or more devices connected to the computer and to analyse and store data signal received from various devices connected to the computer. For example, both V_{G} and V_{DS} values are collected using a computer programme such as LABVIEW. The current response (I_{DS}) from the LGFET is measured and collected from the pico-ammeter and further collated with the LABVIEW programme.

The presence of analytes in the proximity to the SWCNT can affect and modulate the conductance of the SWCNT. For example, the analytes may disturb the electrostatic environment around the SWCNT, thereby affecting the conductance of the nearby carbon nanotube.

A few mechanisms for modulating the conductance of the SWCNT are possible: electrostatic gating, Schottky barrier modulation, capacitance effects, and mobility change, each of which records a characteristic change in the I_{DS}-V_{G} characteristic curve.

Electrostatic gating is associated with a shift in I_{DS}-V_{G} characteristic curve (regardless positive or negative), depending on the charge of the biomolecules bound to the nanotubes.

Modulation of the Schottky barrier is characterized by a decrease in I_{DS} at negative V_{G} bias, and an increase in I_{DS} at positive V_{G} bias.

A change in the capacitance is typified by modulation in the saturation current, whereas mobility changes caused by scattering or other effects of biomolecule/SWCNT interactions show a decrease in gradient under both positive and negative V_{G} bias.

Without wishing to be bound to any particular theory, the small impact or magnitude of the detectable electrical signal may be attributed to the large size difference between the small analyte molecules (typically having size of below 1 kDa) and the complementary coupling analyte-binding molecules (typically having size of above 10 kDa). When an analyte-binding molecule is immobilized on the nanotube surface, the immobilized analyte-binding molecule introduces a large background charge, thus dwarfing the small potential disturbance that the small analyte molecule inflicts upon the analyte:analyte-binding molecule complex formation in the assay detection.

To increase the impact on the detectable signal the small analyte molecule has upon the analyte:analyte-binding molecule complex formation in the assay detection, it has been proposed to couple two or more analyte molecules to each carrier molecule to form an analyte:carrier molecule complex having size comparable to that of the analyte-binding molecule. The specific binding of the analyte-binding molecule with the analyte:carrier molecule complex may introduce significant potential disturbance surrounding the nanotubes, and hence may lead to shifting of the I_{DS}-V_{G} characteristic curve toward the negative or positive V_{G} direction, indicating that the conductance modulation comes primarily from electrostatic gating. This negative (or positive) shift normally is associated with the enrichment of positive (or negative, respectively) charge that results when the analyte:analyte-binding molecule complex is formed.

In order that the invention may be readily understood and put into practical effect, particular embodiments will now be described by way of the following non-limiting examples.

### EXAMPLES

### Example 1

### Fabrication of Biosensor

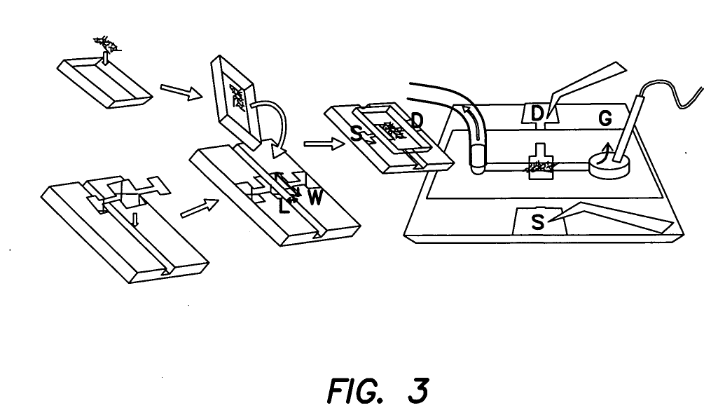

A flexible laminated LGFET was fabricated as follows (FIG. 3): Two slabs of poly(dimethyl siloxane) (“PDMS”) (Sylgard 134, DuPont) were prepared; one slab was molded with a microfluidic channel and the other was featureless. An aqueous dispersion of carboxylated single-walled carbon nanotubes (“SWCNTs”) (2.5 atomic %, Cheaptubes, Inc.) (0.5 mg ml⁻¹) was prepared by dissolving 25 mg of the nanotubes in 50 ml of sodium-dodecyl benzene sulfonate (“SDDBS”) solution (1% w/v). The solution was sonicated for approximately 2 h for homogenization. The aqueous SWCNT solution was then filtered with an alumina filter (Whatman, 0.1 μm) to form a film of nanotubes on the filter. It is to be noted and appreciated that the preparation of the carbon nanotube suspension may be modified for specific improvements. The resulting SWCNT film was then transfer-printed on to the PDMS slabs through a simple stamping step to form the source-drain electrodes in the slabs carrying the fluidic channel, as well as the transistor channel on the flat slab. The two PDMS slabs were then laminated to form the complete device. The source-drain electrodes and the transistor channel were automatically defined by the microfluidic channel with this device architecture.

The electrical contact is made with the aid of syringe needle tip and silver paint. Specifically, the metallic segment of the syringe needle tip is punctured into the dense SWCNT film throughout the thickness of the PDMS to ensure mechanical stability of the contact, and a drop of silver paint is applied at the meeting point of SWCNT film/metal syringe region in fillet configuration to ensure good electrical contact.

### Example 2

### Process Optimisation and Testing of Biosensor

To achieve consistent immuno-sensing response with minimized false signal, a series of background measurements were conducted to ensure reliable biomolecule attachment and process optimization through systematic layer-by-layer analysis.

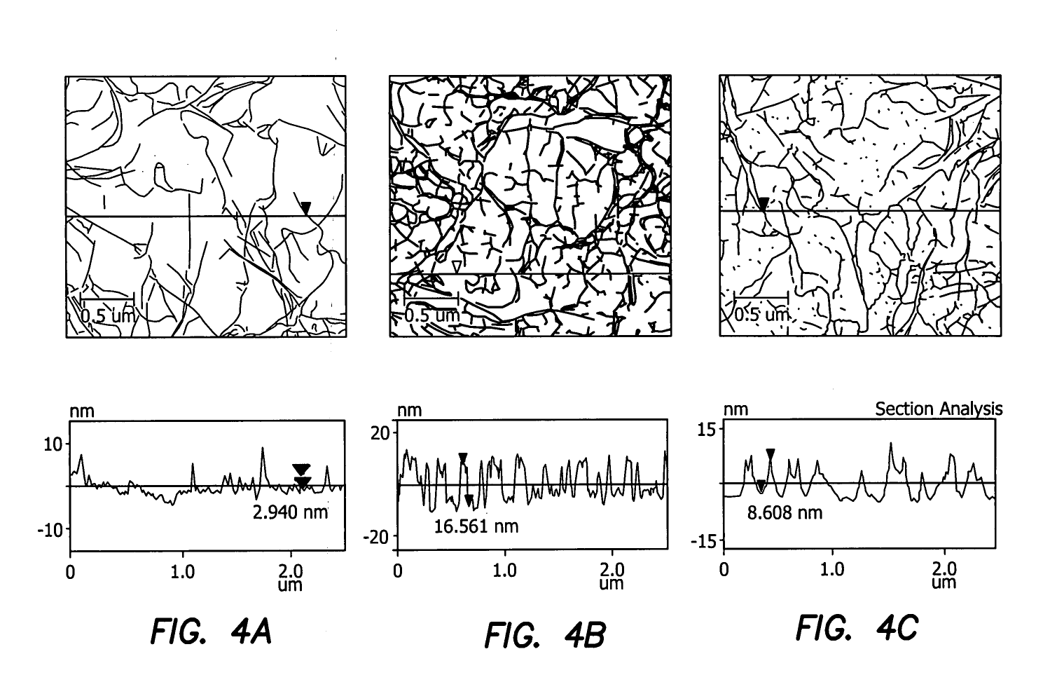

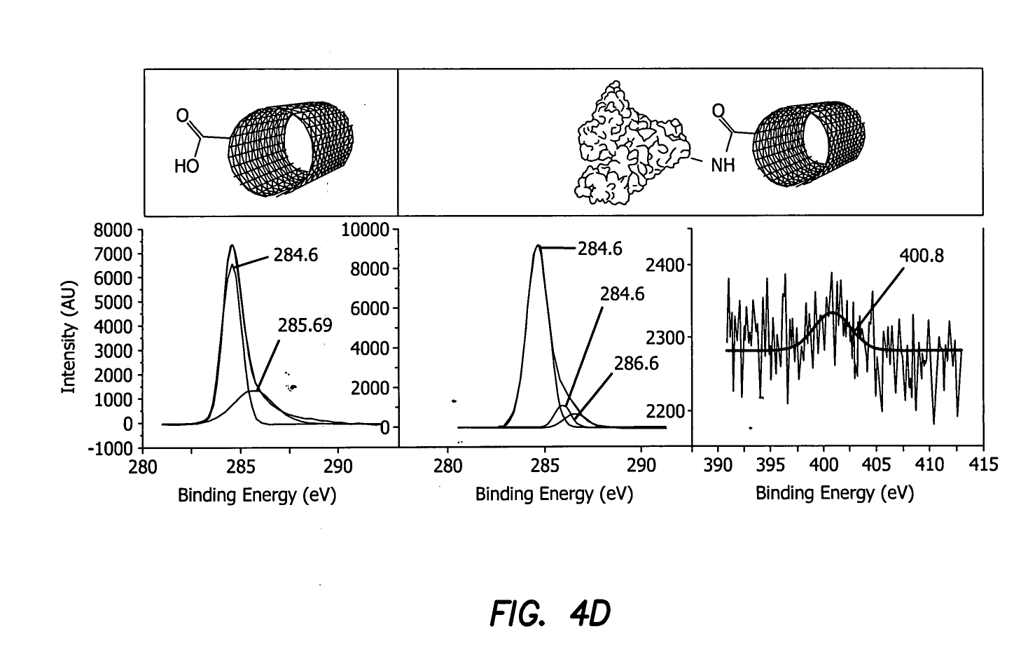

Morphine antibody (“Mor-Ab”) and 6-monoacetylmorphine-bovine serum albumin conjugate (“MAM-BSA”) were shown to interact with carbon nanotube (“CNT”) and height profile analysis compared favourably with the dimensions of Mor-Ab (approximately 10-15 nm) and MAM-BSA (approximately 5-10 nm). FIG. 4 shows the Atomic Force Microscopy (“AFM”) phase profile and its respective height analysis for bare CNT network (FIG. 4(a)), Mor-Ab attachment (FIG. 4(b)) and MAM-BSA attachment (FIG. 4(c)) onto the biosensor. AFM samples were prepared by drop casting 1 mg ml⁻¹ of Mor-Ab and MAM-BSA solution onto a thin CNT random network printed on PDMS substrates respectively and incubation for 1 h, followed by rinsing with PBS and drying with nitrogen gas before imaging. The AFM images show that the biomolecules only bind specifically to the CNT network, without any significant non-specific binding to the PDMS substrate, indicating that biomolecule binding onto the substrate does not interfere with detection. The carbodiimide-activated amidation process for covalently bonding Mor-Ab or MAM-BSA to the CNT surface was verified with X-ray Photoelectron Spectroscopy as shown in FIG. 4(d).

Influential parameters such as pH and ionic strength of electrolyte solution, were optimized for best binding efficacy.

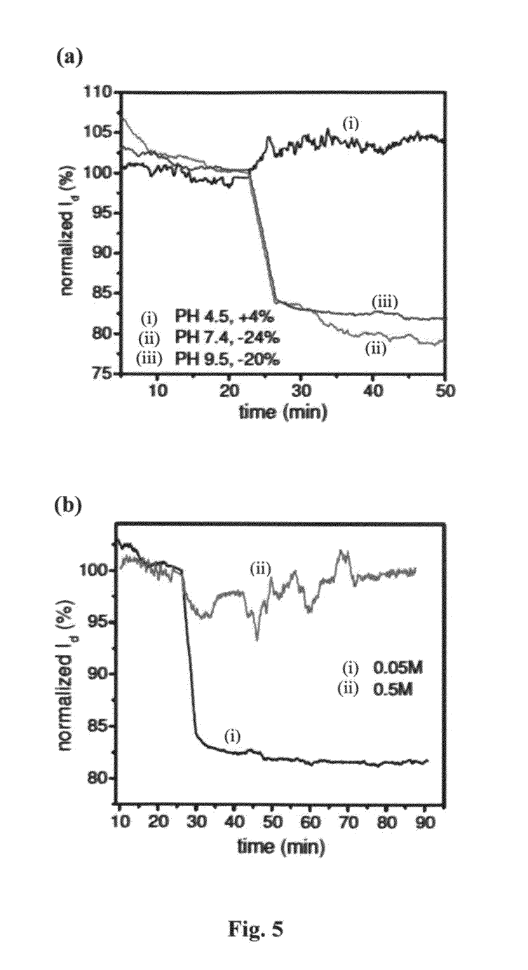

FIG. 5 shows the effects of (a) pH and (b) ionic strength of electrolyte solutions on the conductance of the biosensor. Phosphate buffered saline solutions (“PBS”) of different pH values, i.e. pH 4.5, pH 7.4 and pH 9.5, were tested and changes in the conductance signal were recorded and compared when MAM-BSA conjugate was added in (FIG. 5(a)). Very little electrical signal could be captured at acidic pH 4.5 upon the interaction of SWCNT with MAM-BSA. Detectable electrical signal increased at pH 7.4 and above, producing a significant I_{DS} drop of approximately 20%, indicating the optimal pH range for detection. Similarly, for the ionic concentration study in FIG. 5(b) it was found that a dilution of 10 times of the PBS significantly enhances the sensitivity, which may be attributable to the lesser screening charge, and hence longer debye length for effective charge detection.

As an electrostatically sensitive device, one of the important parameters that influences the device sensitivity is the Debye length (λ_{D}). Upon bio-analyte absorption onto CNT network, the counter ions in the electrolyte solution accumulate near the bio-analyte, causing the electrostatic potential of the absorbed analyte to decay over a length due to the shielding effect which can be represented by the following equation:

$${λ}_{D} = \sqrt{\frac{{ɛ}_{0} {ɛ}_{r} kT}{2 {N}_{A} {e}^{2} I}} \qquad Eqn . ( A )$$

Where I is the ionic strength of the electrolyte in mol m⁻³, ε₀ is the permittivity of free space, εᵣ is the dielectric constant, k is the Boltzmann's constant, T is the absolute temperature in Kelvin, N_{A} is the Avogadro's number and e is the elementary charge. The inverse relationship between Debye length and the ionic strength highlights importance of the ionic-strength of the electrolyte to prevent charge screening effect of the biomolecules so as to allow better detection. In the experiment, ionic concentrations of the PBS were adjusted to 50 mM to lessen the screening charge effect for better sensitivity while maintaining suitable condition for biomolecules.

In addition, the study of the efficiency of various blocking agents (Table 1) suggested the use of 10 v/v % tween-20 (from Sigma-Aldrich) to prevent non-specific binding during the subsequent target recognition step. For comparison, skim milk (from Sigma-Aldrich) and polyethylene glycol (“PEG”) (from Sigma-Aldrich) were used and were found to possess partial blocking capability for the CNT network used.

TABLE 1
Efficiency of Various Blocking Agents

| Blocking agent | ΔI_{ds} (nA) | ΔI_{ds}/I_{ds} (%) | Blocking efficiency |
|---|---|---|---|
| Direct BSA sensing | −8.8 | −8.03 | No blocking |
| 10 v/v % PEG | −10.8 | −2.62 | Less effective |
| 10 v/v % skim milk | +2.9 | +1.26 | Partial blocking |
| 10 v/v % PEG + | +3.3 | +1.80 | Partial blocking |
| 10 v/v % skim milk | | | |
| 10 v/v % Tween-20 | +1.2 | +0.60 | Complete blocking |

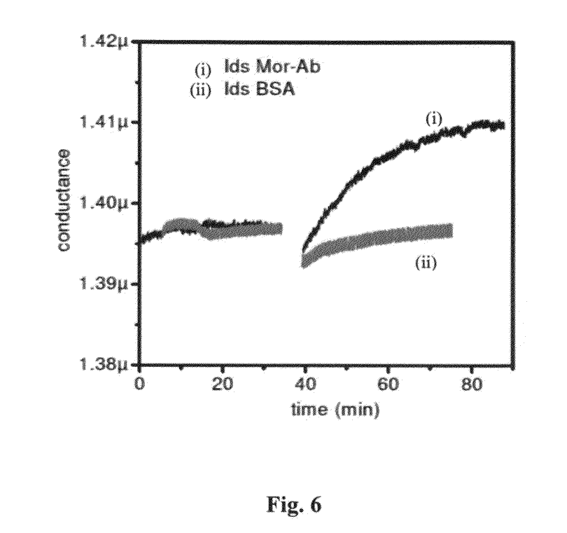

Selectivity study was performed (FIG. 6) to validate and confirm the sensing response after establishing all necessary experimental conditions. The selectivity study shows the specificity of the CNT towards Mor-Ab detection after overnight incubation with MAM-BSA conjugates and blocking with tween-20.

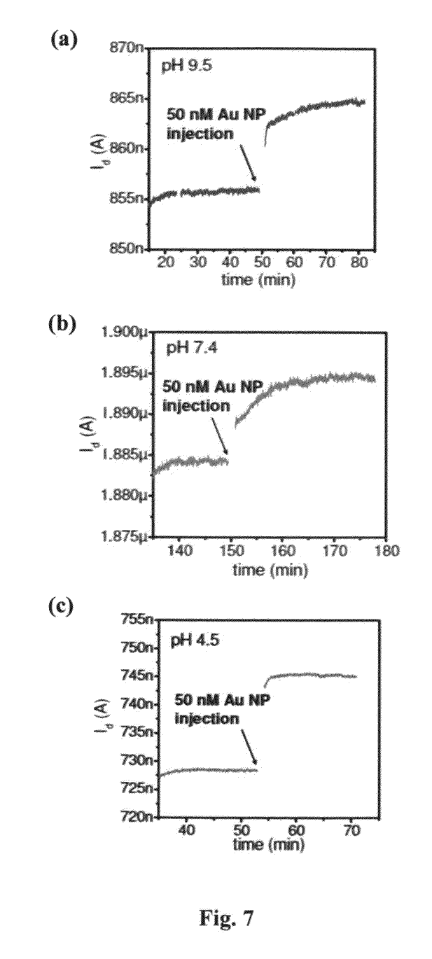

FIG. 7 shows the effect of signal enhancement by gold nanoparticles (50 nM solution) injected into the bare carbon nanotube network of the biosensor at pH 9.5 (FIG. 7(a)), pH 7.4 (FIG. 7(b)), and pH 4.5 (FIG. 7(c)). The results shows that direct gold nanoparticle interaction with CNT triggers the induced hole doping behavior within the experimental window tested from pH 4.5-9.5. I_{DS} increases upon the injection of the gold nanoparticles into the carbon nanotube network. Under all pH conditions tested, signal increment was consistently observed upon the injection, with pH 4.5 giving the highest increment signal based on 3 repetitions. Nevertheless, as it was previously determined that favorable MAM:Mor-Ab binding occurs in the range of pH 7.4-9.5 (FIG. 5(a)), in the following amplication was performed at pH 9.5.

### Example 3

### Detection of Heroin Metabolites

In this example, a competitive immunoassay for the detection of a heroin metabolite (monoacetylemorphine) using a polymer substrate supported carbon nanotubes-based liquid gated transistor was tested. The facile and low cost fabrication process of this biosensor, coupled with its promising detection limit of 15 fg ml⁻¹, demonstrates great potential for adoption for on-site analysis. This ultra sensitive detection of the narcotic metabolite is a consequence of the competitive immunoassay protocol along with the charge enhancement effect of gold nanoparticles which augments the electrostatic perturbance generated from receptor-ligand interaction with the monoacetylmorphine antibodies.

Experimental Details

Materials. Carboxylated SWCNT powder was purchased from Cheaptubes, Inc. PDMS (Sylgard 184) was purchased from Dow Corning, Inc. 6-monoacetylmorphine (“MAM”) was purchased from Cerilliant Analytical Reference Standards. Sodium dodecyl sulfate (“SDS”), tween-20, PEG, skim milk, bovine serum albumin (“BSA”), Freund's complete adjuvant (“FCA”), Freund's incomplete adjuvant (“FIA”), disodium hydrogen phosphate (Na₂HPO₄), and sodium dihydrogen phosphate (NaH₂PO₄) were purchased from Sigma Aldrich. 1-ethyl-3-(3-dimethylaminopropyl)carbodiimide hydrochloride (“EDC”), and N-hydroxysulfosuccinimide (“sulfo-NHS”) were purchased from Pierce Chemicals. Protein-A Sepharose was procured from Amersham Biosciences, India.

Synthesis of Analyte: Carrier Molecule Complex (MAM-BSA Conjugate). The synthesis of carboxylic acid derivative of monoacetylmorphine (MAM-COOH), its bioconjugation with BSA and the generation of anti-morphine antibodies are described in the following: For the synthesis of hapten, the derivatization of MAM was done by refluxing the reaction mixture containing 3 μM of MAM, 24 μM of chloroacetic acid, 45 μM sodium hydroxide and 30 μM acetonitrile for 3 h at 90° C. in an inert nitrogen atmosphere. The presence of a —COOH group was confirmed by thin layer chromatography and infrared spectroscopy. The derivatized hapten (MAM-COOH) was used for the conjugation with BSA using carbodiimide coupling chemistry. For the activation, 50 μM MAM-COOH, 75 μM EDC and 75 μM sulfo-NHS were mixed and incubated for 1 h at room temperature, followed by overnight incubation at 4° C., and centrifuged for 10 min at 10,000×g to remove urea precipitate. For the conjugation of activated hapten with BSA, 30 μM of activated hapten was mixed with 0.15 μM (10 mg) of BSA to prepare the conjugate in a molar ratio of 100:1.

Preparation of Antibodies (Mor-Ab). The antibodies were raised against MAM-BSA conjugate in young six to eight weeks old New Zealand white rabbits. The rabbits were immunized subcutaneously with 250 μg of MAM-BSA mixed with equal volume of FCA at the time of first booster followed by FIA in subsequent booster doses. The rabbits were bleed after the 5^{th} day of each booster and blood was collected, serum precipitated and antibodies (“IgG”) were purified using Protein-A sepharose column. The fractions were then dialyzed against PBS, the IgG concentration was determined at 280 nm and the fractions stored at −20° C. until used.

Synthesis of Signal Enhancer (Gold Nanoparticles) and its Conjugation to Antibody. Monodispersed (30 nm) colloidal gold was prepared by slight modification of Frens method: A 200 mL solution of 0.01% tetrachloroauric acid in Milli-Q water was brought to boiling. 4 mL sodium citrate solution (1% w/v) was added to the boiling gold chloride solution. The solution was allowed to boil for 10 min until it developed the typical bright wine red color of colloidal gold. The average particle size of colloidal gold was determined using transmission electron microscope (Hitachi Model H-7500) operated at 120 kV. The average particle size was determined to be approximately 30±4 nm.

For the preparation of antibody gold conjugate, 90 μg of anti-morphine antibody was prepared in 20 mM phosphate buffer, pH 7.4 and added dropwise into 1 ml colloidal gold solution ([Au]=2.4×10⁻⁴ mol L⁻¹) under mild stirring. The pH of the colloidal gold solution was maintained at 7.4 by addition of 10 mM Na₂CO₃ before adding the antibody. The mixture was incubated overnight at 4° C. and centrifuged at 12,000 rpm for 30 min to remove unconjugated antibody from the solution. The pellet obtained was washed three times with 10 mM Tris (pH 8.0) containing 3% BSA under centrifugation at 12,000 rpm for 30 min to remove traces of unconjugated antibody. The pellet was resuspended in 2 ml of phosphate buffer (20 mM, pH 7.4) and stored at 4° C. before its use. The final concentration of colloidal gold in the antibody-gold conjugate solution was 4.8×10⁻⁴ mol L⁻¹. A Hitachi 2800 UV-vis spectrophotometer was used to measure the absorbance of gold nanoparticles and antibody labeled gold nanoparticles.

Preparation of CNT suspension. To increase the carboxylic functional groups for better molecule attachment, 20 mg of as-purchased carboxylated CNT material was acid treated in 100 ml, 3:1 volume ratio of concentrated H₂SO₄ and HNO₃ and refluxed overnight at 50° C. The refluxed solution was centrifuged several times to separate the tubes from acid solution and neutralize the solution until pH 6. To further enhance tube dispersity, the 1 mg ml⁻¹ treated solution was diluted 10 times with deionised water to a concentration of 0.1 mg ml⁻¹ and 1 w v⁻¹% SDS was added into the final volume. The suspension was sonicated, followed by centrifugation for 1 h at 14,000 rpm to remove the non-dispersed bundles. The extracted supernatant was kept as a stock solution for the entire experiment.

Preparation of Biosensor for Detection. A liquid-gated field effect transistor (“LGFET”) has been fabricated as generally described in previous paragraphs. Carboxylated SWCNT was shown to interact better with BSA. Hence, the acid-treated carboxylated SWCNT was used as the active channel of the LGFET. The carboxylic groups were first activated with EDC and sulfo-NHS for 1 hour at room temperature, rinsed with 50 mM PBS (pH 9.5), followed by 10 μg ml⁻¹ MAM-BSA injection and overnight incubation at 4° C. Excess unbound molecules were removed by rinsing the microchannel with copious amount of PBS, and the device was ready for antibody detection.

Electrical Measurements. Electrical measurements of the CNT-LGFET were performed using a home-built LabView system. For real time monitoring, a liquid gate potential (V_{G}) at −0.5 V was applied to the electrolyte through reference electrode (3M KCl, FLEXREF, World Precision Instruments) and a small drain bias (V_{D}) of 10 mV applied over the source and drain electrodes to obtain the kinetic response at respective detecting steps.

Methodologies for Detecting Analyte

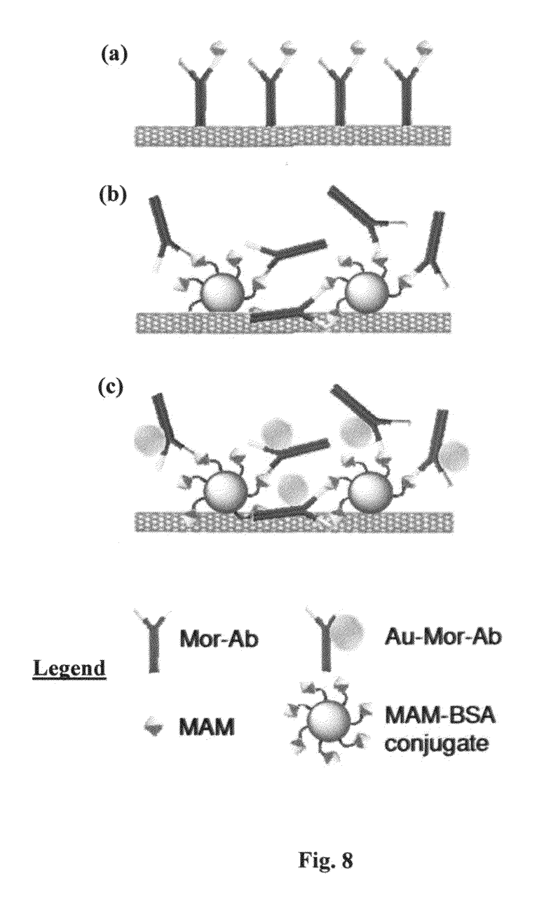

Direct Assay Detection. Mor-Abs were immobilized directly onto the semiconducting channel of the LGFET comprising a network of CNTs (FIG. 8(a)). While the antibody functionalization showed promising change in drain current (I_{DS}) (FIG. 9(a)), no detectable signal was observed upon injection of 1 μg ml⁻¹ of MAM. This observation is explained by investigating the underlying sensing mechanism which may include electrostatic gating, work function modulation, capacitance effect, and mobility change. Electrostatic gating is correlated with a threshold voltage shift; whereas Schottky barrier modulation is characterized by a decrease in I_{DS} at gate voltage (V_{G})<0 and increase in I_{DS} at V_{G}>0. Capacitance effect and mobility changes manifest themselves in a change in transconductance and decrease in I_{DS} respectively.

Attachment of the Mor-Ab onto CNT causes a positive threshold voltage shift (FIG. 9(a)) with the relatively unchanged transconductance. This indicates that the active sensing mechanism is related to the electrostatic gating effect, wherein the highly negatively-charged Mor-Ab induces positive charges in the CNTs thus shifting the I_{DS}-V_{G} curve towards positive gate voltages and leading to an increase in I_{DS}.

Although the Mor-Ab causes a threshold voltage shift of approximately +50 mV, the subsequent specific binding with MAM is not detected (i.e. no change in I_{DS}-V_{G} curve as shown in FIG. 9(a)). The insensitivity is attributable to the relative sizes of MAM (approximately 327 Da) and Mor-Ab (approximately 150 kDa), where the electrostatic charge of the MAM is screened by the large Mor-Ab molecules. Owing to this screening, MAM has little effect on the electrostatic environment of the semiconducting CNT network.

Reverse Assay Detection. MAM-BSA conjugates were immobilized directly onto the semiconducting channel of the LGFET comprising a network of CNTs and Mor-Ab molecules were subsequently introduced (FIG. 8(b)). FIG. 9(b) shows an increase in I_{DS} of approximately 40 nA with injection of Mor-Ab concentration of 1 μg ml⁻¹. This result supports the above hypothesis of the screening effect of small analyte by large Mor-Ab molecules. By conjugating a large BSA carrier (approximately 66.4 kDa) to small analyte MAM, the size difference between MAM-BSA conjugate and Mor-Ab (approximately 150 kDa) is reduced, and hence the electrostatic charge disturbance inflicted by the MAM-BSA conjugate upon binding to Mor-Ab is detected.

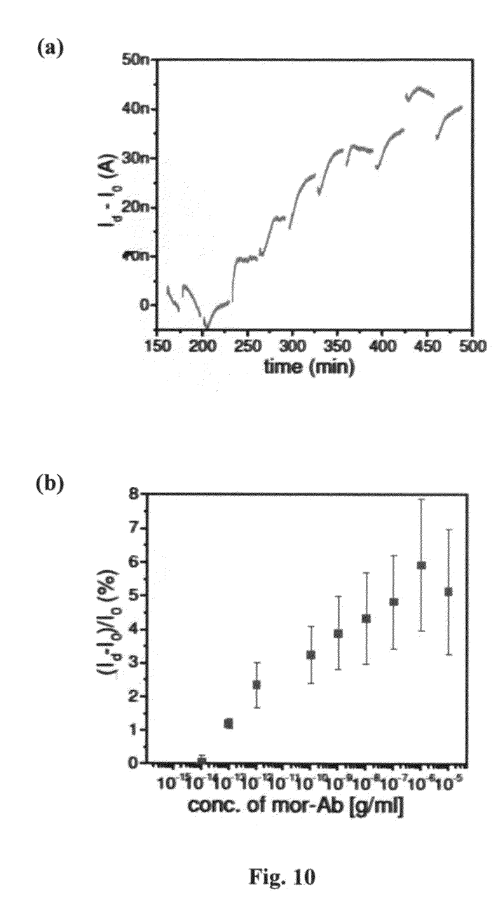

FIG. 10 shows (a) kinetic measurements and (b) concentration study of the detection scheme of FIG. 8(b), with concentrations varying from 10 fg ml⁻¹, 100 fg ml⁻¹, 1 pg ml⁻¹, 100 pg ml⁻¹, 1 ng ml⁻¹, 10 ng ml⁻¹, 100 ng ml⁻¹, 1 μg ml⁻¹, and 10 μg ml⁻¹, respectively with three to five repetitions. The kinetic measurements show an increasing I_{DS} with increasing Mor-Ab injected into the CNT microchannel which binds the MAM-BSA conjugates. The I_{DS} increment signifies an induced positive doping was contributed to the CNT microchannel upon localized binding interaction of Mor-Ab with MAM-BSA conjugate. The limit of detection (formula 3σ/S) of the detection scheme of FIG. 8(b) can be estimated at approximately 100 fg ml⁻¹, using the slope (S) and standard error (σ) extracted from the linear fitted line.

Reverse Assay Detection with Gold Nanoparticle Conjugated to Antibody. MAM-BSA conjugates were immobilized directly onto the semiconducting channel of the LGFET comprising a network of CNTs and Au-Mor-Ab conjugates were subsequently introduced (FIG. 8(c)). The electrostatic charge signal of the reverse assay detection without conjugated gold nanoparticles is now amplified with the use of gold nanoparticles conjugated to Mor-Ab. Gold nanoparticles offer an interesting route to ascertain the underlying detecting mechanism because gold nanoparticles typically carry negative charge as a result of the citrate ions absorbed during the nanoparticles synthesis through citrate reduction method. Hence, if the detecting mechanism in the biosensor is dominated by electrostatic gating, addition of gold nanoparticles leads to positive doping of the channel hole concentration. The positive charge enhancement effect will be superimposed on the MAM-BSA:Mor-Ab binding interaction, thus resulting in larger signal amplification.

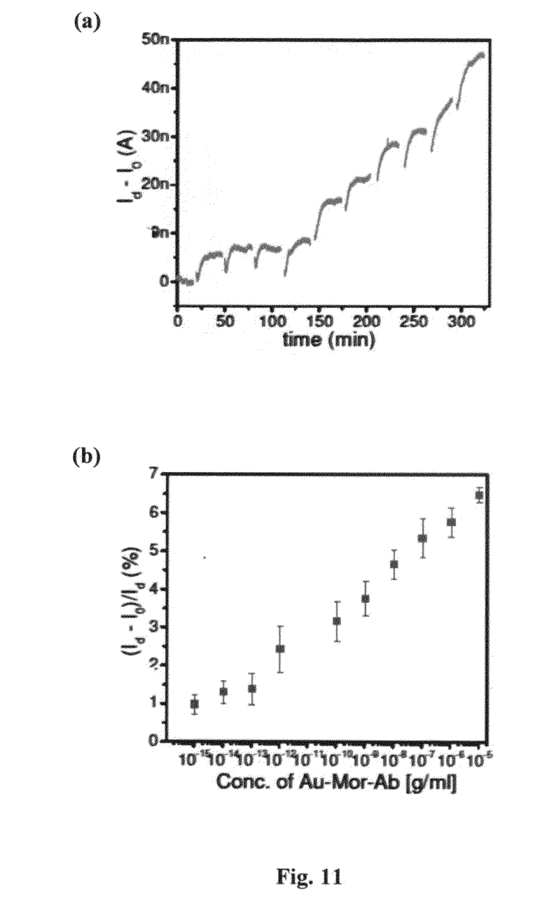

FIG. 11 shows (a) kinetic measurements and (b) concentration study of the detection scheme of FIG. 8(c), with concentrations varying from 1 fg ml⁻¹, 10 fg ml⁻¹, 100 fg ml⁻¹, 1 pg ml⁻¹, 100 pg ml⁻¹, 1 ng ml⁻¹, 10 ng ml⁻¹, 100 ng ml⁻¹, 1 μg ml⁻¹, and 10 μg ml⁻¹, respectively with three to five repetitions. The limit of detection was shown to have improved by two orders of magnitude to approximately 1.2 fg ml⁻¹. The study signifies that labeling of gold nanoparticles onto Mor-Ab can act as a signal amplification tool in morphine detection. It is further observed that with the aid of the nanoparticles, the signal is more stable with smaller range of data fluctuations, hence pushing the limit of detection by two orders of magnitude from 130 fg ml⁻¹ to 1.2 fg ml⁻¹. It is to be noted and appreciated that since the Mor-Ab shows a net negative charge, negatively-charged gold nanoparticles are a convenient scheme for signal amplification. For detection of positively-charged biomolecules, for example poly-L-lysine, positively-charged nanoparticles would be expected to give corresponding signal amplification.

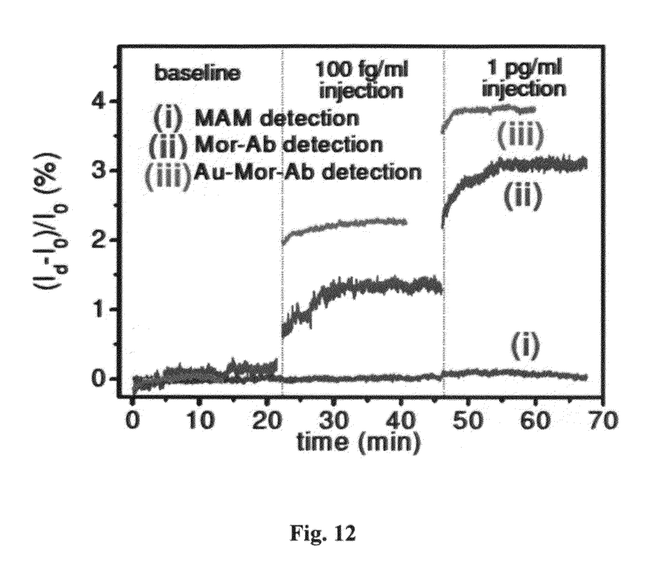

FIG. 12 summarizes the kinetic measurements for the above three detection schemes. For each respective measurement, data normalized with respect to the baseline value (i.e. conductance signal in blank PBS after blocking) upon injections of MAM, Mor-Ab, and Au-Mor-Ab, respectively, are presented. From the respective kinetic curves shown, it is evident that signal detection had improved drastically with the conjugation of MAM to BSA carrier, and further signal amplification followed after conjugation of gold nanoparticles to Mor-Ab.

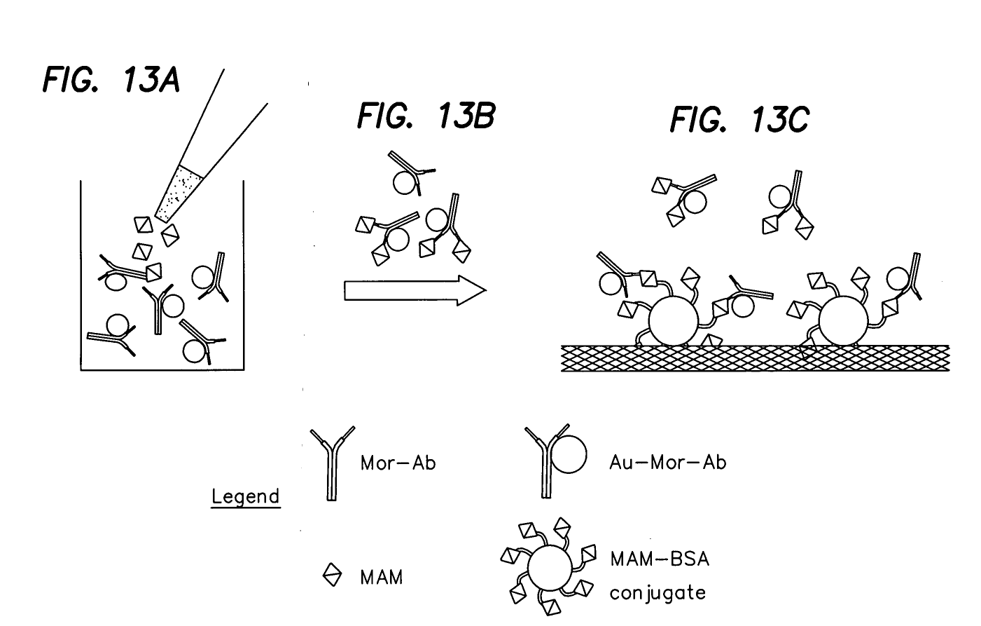

Competitive Assay Detection. As discussed in previous paragraphs, the direct assay scheme (FIG. 8(a)) was not able to detect MAM due to the screening effect of the large Mor-Ab molecule. Therefore, a competitive assay detection scheme was developed for the detection of MAM (FIG. 13). In this competitive scheme, a fixed concentration (1 μg ml⁻¹) of Au-Mor-Ab was added to varying MAM concentrations (1 fg ml⁻¹ to 1 μg ml⁻¹) (FIG. 13(a)) and incubated for approximately 1 h to allow sufficient interaction (FIG. 13(b)), which results in a mixture of Au-Mor-Ab:MAM conjugates and excess free Au-Mor-Ab molecules. As some Au-Mor-Ab have already interacted with MAM, only the free Au-Mor-Ab can be bound to the MAM-BSA conjugate, leading to an increase in I_{DS}. Upon injection into the LGFET, these free Au-Mor-Ab compete (with other moieties in the solution) for binding with the MAM-BSA conjugates (FIG. 13(c)).

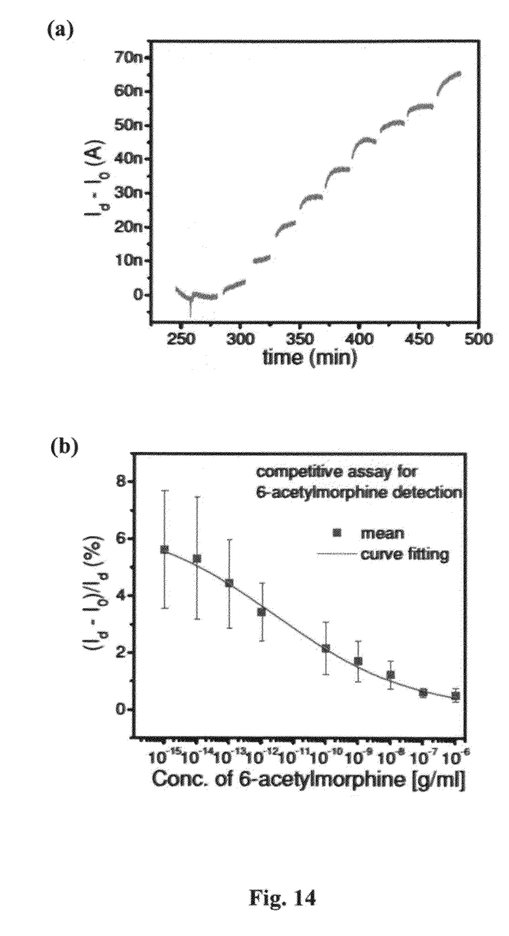

FIG. 14 shows the (a) kinetic measurements and (b) concentration study of the competitive detection scheme of FIG. 13, with concentrations varying from 1 μg ml⁻¹, 100 ng ml⁻¹, 10 ng ml⁻¹, 1 ng ml⁻¹, 100 pg ml⁻¹, 1 pg ml⁻¹, 100 fg ml⁻¹, 10 fg ml⁻¹, and 1 fg ml⁻¹, respectively.

The binding between the free Au-Mor-Ab and MAM-BSA conjugate leads to an I_{DS} increment (FIG. 14(a)), similar to that observed in FIG. 11(a). The larger the starting concentration of MAM, the fewer the free Au-Mor-Ab are available for binding with MAM-BSA conjugates since the probability of Au-Mor-Ab interacting with MAM prior to injecting into the LGFET is higher with larger MAM concentration. Consequently, the concentration plot (FIG. 14(b)) of the competitive scheme is inversely proportional to the concentration of MAM. The limit of detection yielded in this competitive scheme is estimated to be approximately 15 fg ml⁻¹, which is poorer than the Au-Mor-Ab:MAM-BSA detection scheme of FIG. 11(b) although both detection schemes rely on similar specific binding interactions. This decrease in sensitivity and larger signal fluctuation for competitive detection scheme may be attributed to the background signal and screening charge effect caused by the inactive Au-Mor-Ab:MAM moiety in the solution.

### Example 4

### Detection of 2,4-dichlorophenoxyacetic Acid Herbicides in Soil Samples

In this example, a laminated SWCNT LGFET microfluidic-based biosensor and methodologies, direct, reverse and competitive immunoassays, for the detection of a commonly used herbicide 2,4-dichlorophenoxyacetic acid (“2,4-D”) in liquid environment is illustrated. The LGFET is made entirely of plastic, is low cost and the instrumentation required for detecting can be made field-deployable. The detection as extracted from the drain current response of the LGFETs yielded a real-time, label-free detection of free 2,4-D molecules with extremely low detection limits of approximately 500 fM (i.e. 0.5 part-per-trillion) and 50 pM (i.e. 50 part-per-trillion) in soil extract and buffer solution, respectively. These detection limits are much lower than the maximum residual limit of 500 nM (i.e. 500 part-per-billion) for the 2,4-D established by the World Health Organisation in drinking water.

Experimental Details

Materials. Technical grade 2,4-D, BSA, N-hydroxysuccinimide ester (“NHS”), N′,N″dicyclohexylcarbodiimide (“DCC”), dimethyl formamide (“DMF”), keyhole limpet hemocyanine (“KLH”), FCA, FIA, goat anti-rabbit IgG-HRP conjugate, trifluoroacetic acid (“TFA”) were purchased from Sigma-Aldrich. 3,3,5,5′-tetramethylbenzidine (“TMB”) was purchased from Bangalore Genei. Protein A sepharose and sepharose 4 B were procured from Pharmacia. Enzyme-linked immunosorbent assay (“ELISA”) plates (Nunc F96 MicroWell) were obtained from Maxisorp. Na₂HPO₄ and NaH₂PO₄ were purchased from Merck. Tween-20 blocking agent and sodium-dodecyl benzene sulfonate (“SDDBS”) were purchased from Sigma-Aldrich. PDMS (Sylgard 134) was purchased from DuPont, and carboxylated SWCNTs (2.5 atomic %) were purchased from Cheaptubes, Inc.

Preparation of Antibody (anti-(2,4-D)). The preparation of antibody against 2,4-D (“anti-(2,4-D)”) is was carried out as follows: The hapten was first prepared by covalently binding the BSA to the 2,4-D with the assistance of DCC+NHS carbodiimide linker molecules, which forms an amide bond between the carboxylic group of the 2,4-D and the surface amino group of the protein. Additionally, the 2,4-D was also conjugated with KLH protein for the coating of ELISA plates with a similar linking strategy, followed by purification of the conjugates using a P10 gel filtration column (Pharmacia). The fractions with maximum protein concentration were collected and stored in aliquots at −20° C. Subsequently for raising antibodies, six- to eight-weeks old New Zealand white rabbits were immunized subcutaneously with 300 to 400 μg of the 2,4-D-BSA conjugate emulsified in FCA. Succeeding booster doses were given with FIA at the interval of 21 days. Afterward, the blood was collected and the serum was precipitated. The immunoglobulin (“IgG”) fractions were purified from the sera using protein-A sepharose column and the bound antibodies were eluted with 0.1M glycine-HCl buffer (pH 2.5). The eluted IgG fractions were pooled and dialyzed against phosphate buffered saline (“PBS”) (pH 7.4) at 4° C., and stored in aliquots at −20° C. The protocol engaged for generation of anti-(2,4-D) from the laboratory animal as reported herein complies with the relevant laws and institutional guidelines, as approved by the Animal Ethics Committee of Institute of Microbial Technology (India) prior to conducting the experiment.

Preparation of Soil Samples. Soil samples were obtained from a local plant nursery. To prepare the soil extract, one gram of soil was dispersed in a 50 ml deionised water (Millipore). The suspension was then sonicated for 2 h followed by centrifugation at 3000 rpm for 30 min to obtain the soil extract.

Electrical Measurements and Detection. Transistor measurements were performed by applying a gate bias (V_{G}) with a function generator (Thurlby Thandar Instrument, TTi TG1304), connected to Ag/AgCl reference electrode (World Precision Instruments). The V_{G} applied was also monitored with a digital oscilloscope (Agilent DS03062A). A voltage source/pico-ampere meter module (Keithley 6487) was used to supply the source-drain bias (V_{DS} 10 mV) which measures the source-drain current (I_{DS}) at the same time. All the electrical instruments were synchronized by a personal computer through a general purpose interfacing bus (GPIB) card (National Instrument) and a programming code written in LabVIEW 7.1.

Detection of 2,4-D and anti-(2,4-D) antibody was performed through I_{DS}-V_{G} measurement. To perform direct immuno detection of the 2,4-D, the corresponding antibodies (5 μM) were first covalently attached to the nanotubes to act as the analyte-binding molecule via DCC+NHS coupling chemistry. Then, free unconjugated 2,4-D (5 μM) was introduced into the microfluidic channel while the I_{DS}-V_{G} signal was recorded. Similarly, in the case of reverse detection of anti-(2,4-D), the nanotube surface was first incubated with 2,4-D-BSA conjugate (5 μM) and subsequently, the device was exposed to the antibodies solution (5 μM). The immunocomplex formation in both assays was detected by monitoring the I_{DS}-V_{G} curve before and after the immunoreaction.

Additionally, kinetic measurements were also performed in triplicate for: (1) reverse immunoassay for the detection of anti-(2,4-D) in PBS buffer (pH 7.4, 7.3 mS cm⁻¹), (2) competitive immunoassay for the detection of the free 2,4-D in PBS buffer (pH 7.4, 7.3 mS cm⁻¹), and (3) real sample analysis for the detection of the free 2,4-D by competitive immunoreaction in soil extract (pH 7, 12 μS cm⁻¹). In all three cases, the 2,4-D-BSA conjugate was used as the analyte-binding molecule to capture the anti-(2,4-D) antibodies. Logarithmic serial dilutions (5 fM up to 500 μM) of 2,4-D or anti-(2,4-D) were prepared to determine the limit of detection of LGFET. In the reverse immunoassay, anti-(2,4-D) antibodies at different concentrations were directly applied to the LGFET, which had been initially functionalized with the 2,4-D-BSA conjugate. In the case of competitive immunoassay, a fixed concentration of antibody (50 μM) was first incubated with different concentrations of 2,4-D for 2 h at room temperature (standard solution or soil extract), and subsequently employed for sensitivity studies using LGFET. Selectivity of the device was investigated by exposing a non-pairing protein, such as native BSA to the 2,4-D-BSA conjugate while monitoring the I_{DS}. All measurements were done in triplicates to determine the reproducibility and the standard deviation of the data.

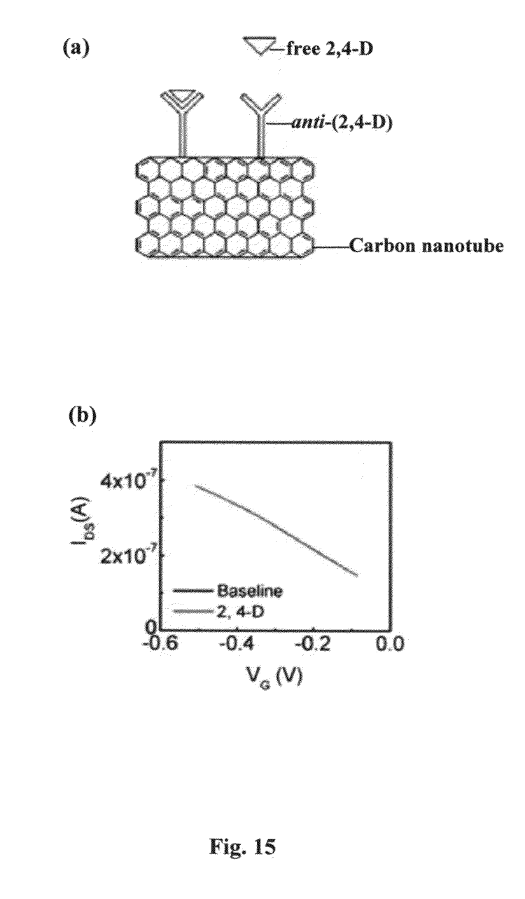

In the conventional direct assay detection scheme illustrated in FIG. 15(a), the analyte-binding molecule (anti-(2,4-D)) is bound to the nanotube surface, while the free 2,4-D in solution functions as the analyte. The baseline in transfer characteristic shown in FIG. 15(b) is recorded after the antibody has been bound to the nanotube surface, and the unbound SWCNT surface has been passivated with the tween-20 blocking agent. The I_{DS}-V_{G} characteristic curves before and after the 2,4-D exposure completely overlap, indicating that the biosensor is not able to detect the immunocomplex formation.

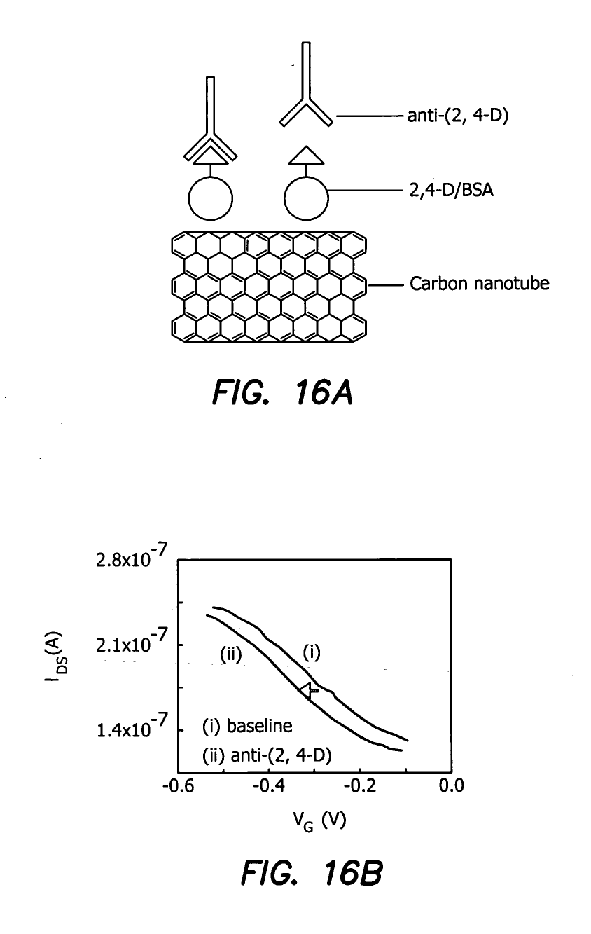

In the present reverse immunoassay detection scheme of anti-(2,4-D) illustrated in FIG. 16(a), the 2,4-D-BSA conjugate functions as the analyte:carrier molecule complex and is bound to the nanotube surface, while the 2,4-D antibody acts as the analyte-binding molecule. In contrast to the results obtained in the direct immunoassay detection scheme illustrated in FIG. 15(b), the I_{DS}-V_{G} characteristic curve now displays a considerable shift toward the negative V_{G} values shown in FIG. 16(b).

The differing response levels may be attributed to the large size difference between the free 2,4-D (having molecular mass 221.04 Da or g mol⁻¹) and the anti-(2,4-D) (having molecular mass 15 kDa or g mol⁻¹). When anti-(2,4-D) is bound to the nanotube surface, the bound anti-(2,4-D) introduces a large background charge, thus dwarfing the small potential disturbance that the free 2,4-D inflicts upon the immuno-complex formation in the conventional direct immunoassay detection.

In contrast, in the present reverse immunoassay detection, the binding of anti-(2,4-D) with the 2,4-D-BSA conjugate introduces significant potential disturbance surrounding the nanotubes, and hence leads to shifting of the I_{DS}-V_{G} characteristic curve toward the negative V_{G} direction shown in FIG. 16(b), indicating that the conductance modulation comes primarily from electrostatic gating. This negative shift normally is associated with the enrichment of positive charge that results when the immunocomplex is formed.

In general, possible sensing mechanisms in LGFETs include electrostatic gating, Schottky barrier, capacitance effect, and mobility modulation; each of which records a characteristic change in the I_{DS}-V_{G} characteristic curve. Electrostatic gating is associated with a shift in I_{DS}-V_{G} characteristic curve (regardless positive or negative), depending on the charge of the biomolecules bound to the nanotubes.

It can be inferred and concluded from the I_{DS}-V_{G} shift in FIG. 16(b) that the immunocomplex primarily influences the conductance of the nanotube through electrostatic gating modulation. Furthermore, the gradient of I_{DS}-V_{G} curve does not seem to be strongly affected by the immunocomplex formation, indicating that the charge-carrier mobility in the nanotube is unchanged following the immunoreaction.

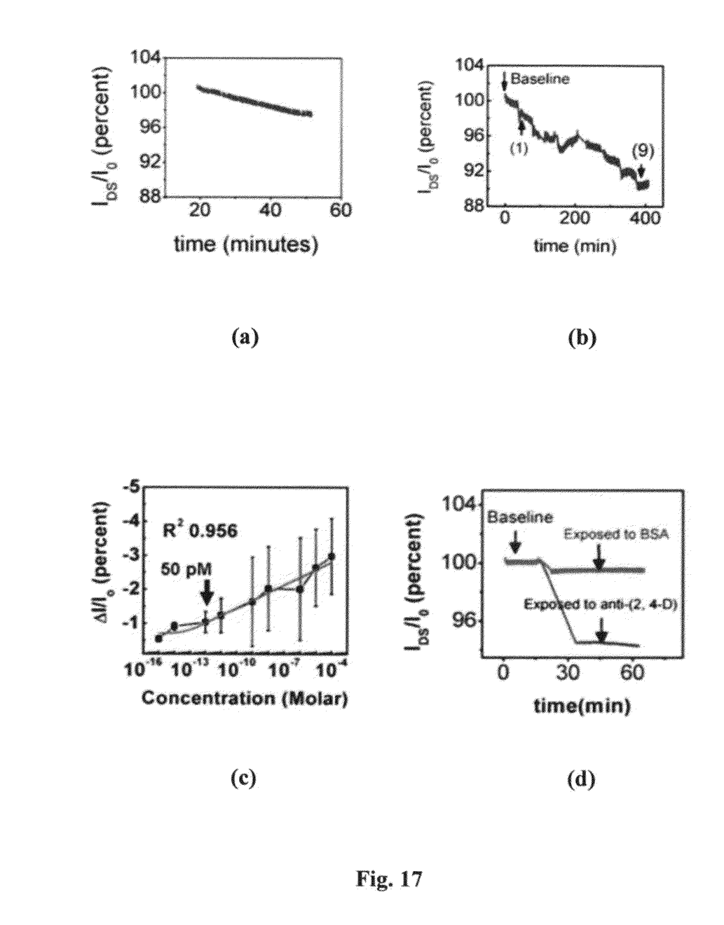

Prior to the real-time kinetic measurement, the drift characteristic of the LGFET was taken in blank PBS buffer as shown in FIG. 17(a). This drift usually is removed off-line before performing analysis to obtain the calibration plot from the kinetic measurement data. The immunocomplex formation in real time was probed by using the LGFET in kinetic mode, which monitors the I_{DS} as a function of time at a fixed V_{G} (−500 mV) and V_{DS} (10 mV). Hence, in this regard, the non-symmetric I_{DS}-V_{G} response as observed in FIG. 15(b) and FIG. 16(b) is also reflected in the kinetic data, because a shift toward negative V_{G} bias in the I_{DS}-V_{G} corresponds to a I_{DS} drop in the kinetic measurement.

In the first set, we have tested the device for detection of the antibody (in PBS buffer pH 7.4, 7.3 mS cm⁻¹) in reverse immunoassay format. In this case, the 2,4-D-BSA conjugate was first covalently immobilized on to the SWCNT using standard carbodiimide linker chemistry. The LGFET was then employed to capture free antibody in the solution, while measuring the I_{DS} in real-time and keeping the V_{G} values constant. FIG. 17(b) reveals the kinetic response from the prepared device upon exposure to anti-(2,4-D) solution at increasing concentrations. The I_{DS} here has been normalized to the baseline value (I₀) to remove variability that comes from different numbers of nanotubes on the device. The decrease in I_{DS} indicates that the immunocomplex formation results in enrichment of the positive charge surrounding the nanotube, which is also in support of the data shown in FIG. 16(b). This positive charge can be attributed to basic amino acid residues, which usually populates the binding pocket of antibodies.

Expectedly, this drop in I_{DS} becomes larger as the antibody concentration in the solution is increased (FIG. 17(b)). The positive correlation between the I_{DS} drop and the anti-(2,4-D) concentration is characteristic of the reverse immunoassay tests, because the signal level is directly proportional to the concentration of the antibodies.

The general trend of the I_{DS} drop at different anti-(2,4-D) concentrations is noticeable in the calibration curve (FIG. 17(c)) which represents three kinetic measurement replicates. The calibration data was fitted by the regression equation:

$$\frac{{I}_{DS}}{{I}_{o}} = a - b log ( [ anti - ( 2 , 4 - D ) ] + c ) \qquad ( Eqn 1 )$$

yielding a correlation factor R²=0.957 indicating a good fit between the regression and the underlying data points. The sensitivity of the LGFET in Eqn. (1) is associated with its gradient, which equals to approximately 0.21% decade⁻¹, and estimated detection limit of approximately. 50 pM (i.e. 50 part-per-trillion) for the case of detection of anti-(2,4-D) in PBS buffer.

The selectivity of the LGFET was also investigated by exposing the device toward a non-pairing protein, such as native BSA. FIG. 17(d) shows that the I_{DS} drop only slightly upon exposure to the native BSA, also confirming the effectiveness of tween-20 as the blocking agent which prevents attachment of excess biomolecules directly to CNTs and potentially interfering with the sensing response.

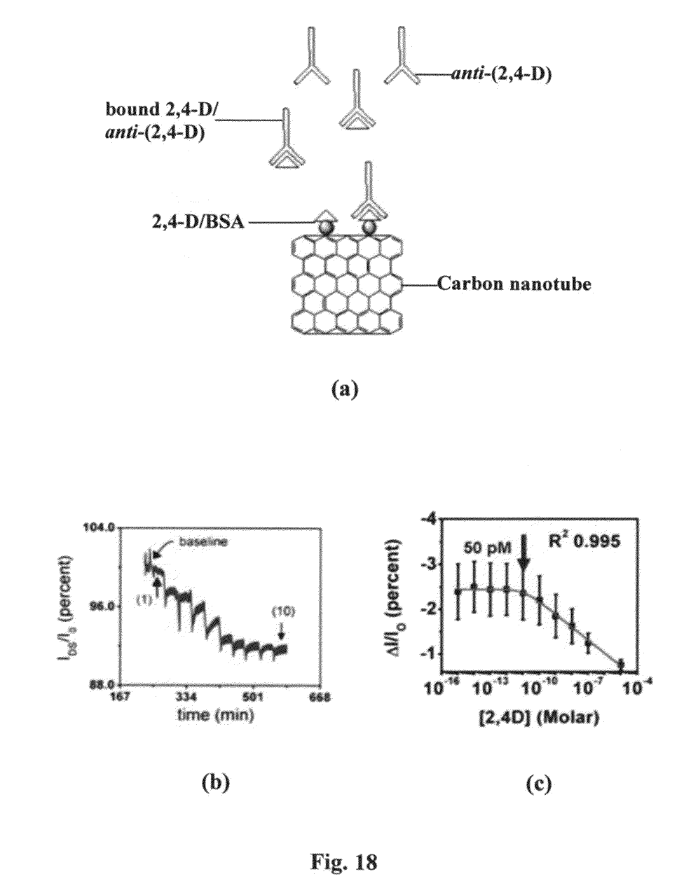

As discussed earlier (FIG. 15(b)), the detection of free 2,4-D using direct immunoassay protocol is not possible due to its small size as compared to the anti-(2,4-D) which results in negligible electrostatic changes produced by the antigen molecule. An alternative to detect the free 2,4-D in solutions is through a competitive immunoassay protocol. For this, a fixed concentration of antibody (50 μM) was mixed with a series of free 2,4-D solutions differing in concentration. Upon injection into the LGFET, the free 2,4-D molecule competes with the 2,4-D-BSA conjugate to bind with the anti-(2,4-D) in solution (FIG. 18(a)). Therefore, the larger the concentrations of free 2,4-D, the less the free binding sites of anti-(2,4-D) that bind with the 2,4-D-BSA conjugate. Hence, the competitive assay protocol yields a calibration plot that is inversely proportional to the concentration of free 2,4-D in solution. The kinetic response (FIG. 18(b)), however, is similar to the reverse immunoassay protocol shown in FIG. 17(b), because in both cases, the device detects binding of anti-(2,4-D) to the 2,4-D-BSA conjugate.

The inverse relationship between current and concentration of 2,4-D is also observed in the calibration curve (FIG. 18(c)) which is derived from three kinetic measurement replicates. As described, earlier in Eqn. (1), the R²=0.995 indicates excellent regression fit with sensitivity of approximately 0.33% decade⁻¹, and a 2,4-D detection limit of approximately 50 pM (i.e. 50 part-per-trillion), which is much lower than the World Health Organization established maximum residual level of 2,4-D concentration in drinking water at 500 nM (i.e. 500 part-per-billion) levels.

In a third set of experiments, the competitive immunoassay was employed to detect the free 2,4-D in the real sample of soil extract with the LGFET. The pH and the conductivity of the soil extract were found to be in the range of 7, and 12 μS cm⁻¹, respectively. This indicates that even though the soil extract has a pH, similar to that of lab condition (pH 7.4, conductivity 7.3 mS cm⁻¹), the ionic-strength of the extract is much lower than those tested in the lab.

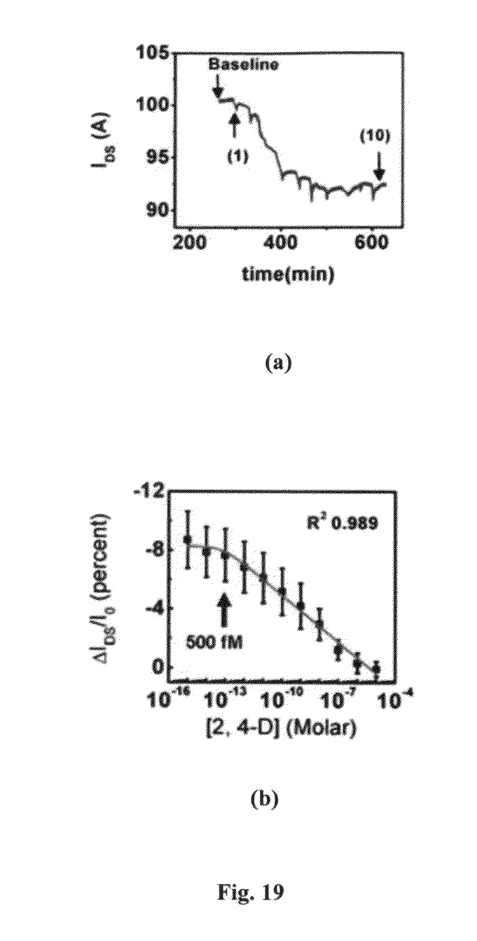

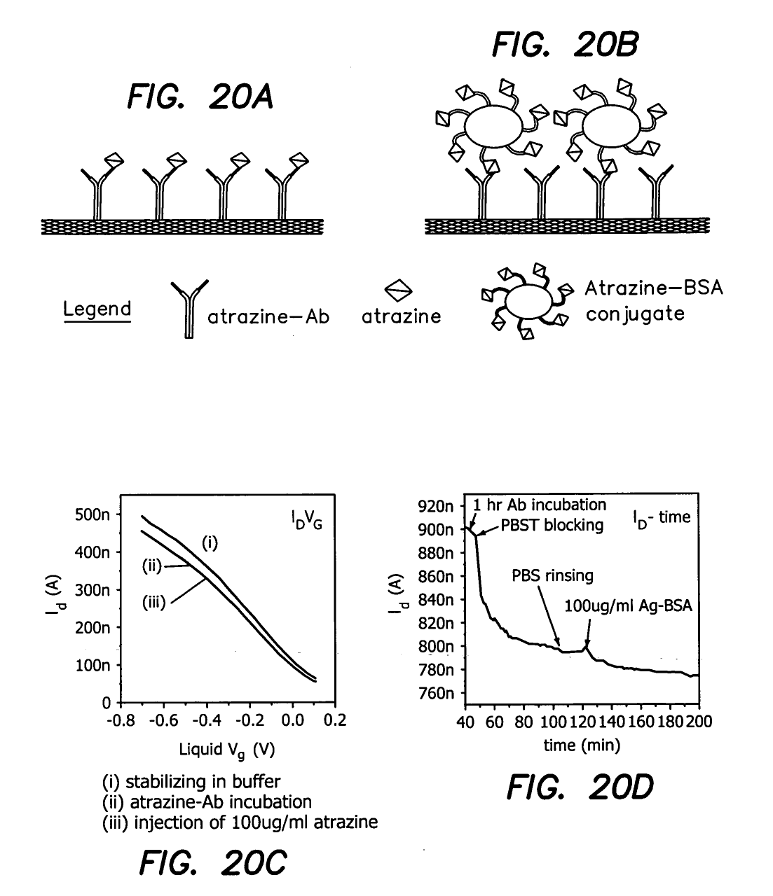

From the kinetic response and calibration plots (FIG. 19(a), FIG. 19(b)) is can be seen that the change in I_{DS} is almost similar to those observed in FIG. 18(b) and FIG. 18(c), except that it is somewhat larger in the case of soil extract. This slight amplification is attributable to the lower ionic-strength (12 μS cm⁻¹) of the soil extract as compared to PBS buffer solution (7.3 mS cm⁻¹). In aqueous solutions, the polar and/or charged surface of biomolecules attracts counter-ions from their surroundings. These counterions screen the stray potential which originates from the biomolecules and is required to disturb the conductance of the SWCNTs. In higher ionic-strength solutions, this screening process is more effective than in lower-ionic strength and hence, the signal in LGFETs is inversely proportional to the ionic strength of the solution, and probably is the reason why the change in I_{DS} is slightly larger in the case of soil extract than in PBS buffer.

The correlation factor R² of 0.989 as extracted from Eqn. (1) and the sensitivity of 0.94% decade⁻¹ is higher than the previous two cases and the limit of detection is estimated at 500 fM (i.e. 0.5 part-per-trillion). The improvement in the sensitivity and limit of detection over the case of 2,4-D detection in PBS buffer is a direct consequence of the larger I_{DS} drop observed in the soil extract.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by specific embodiments and optional features, modification and variation of the inventions embodied therein herein disclosed may be resorted to by those skilled in the art, and that such modifications and variations are considered to be within the scope of this invention.

The content of all documents cited herein is incorporated by reference in their entirety.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are in the following claims. In addition, where features or aspects of the invention are described in terms of Markush groups, those skilled in the art will recognize that the invention is also thereby described in terms of any individual member or subgroup of members of the Markush group.

1. A method for detecting the presence or amount of an analyte, said method comprising:
(a) coupling the analyte to a carrier molecule, wherein the carrier molecule is larger in size, electrically charged and/or polar, to form an analyte:carrier molecule complex;
(b) contacting the analyte:carrier molecule complex of (a) with an analyte-binding molecule coupled to a semiconducting nanostructure; and
(c) determining the change in conductance upon binding of the analyte:carrier molecule complex to the analyte-binding molecule and correlating the determined change in conductance to the presence or amount of the analyte.

2. The method of claim 1, wherein the carrier molecule is conjugated to a signal enhancer.

3. The method of claim 2, wherein the signal enhancer is selected from the group consisting of a metal nanoparticle, a quantum dot, a carbon-based nanomaterial, a silicon particle, a silica particle, an organic molecule, and a mixture thereof.

4. A method for detecting the presence or amount of an analyte, said method comprising:
(a) coupling the analyte to a carrier molecule, wherein the carrier molecule is larger in size, electrically charged and/or polar, to form an analyte:carrier molecule complex;
(b) immobilizing the analyte:carrier molecule complex of (a) on a semiconducting nanostructure;
(c) contacting the immobilized analyte:carrier molecule complex with an analyte-binding molecule; and
(d) determining the change in conductance upon binding of the analyte-binding molecule to the immobilized analyte:carrier molecule complex and correlating the determined change in conductance to the presence or amount of the analyte.

5. The method of claim 4, wherein in (a) a defined amount of analyte is used and prior to (c) a defined amount of analyte-binding molecules is contacted with an unknown amount of the analyte to form a mixture of free analyte-binding molecules and analyte-bound analyte-binding molecules, wherein in (d) the change of conductance upon binding of the free analyte-binding molecules to the immobilized analyte:carrier molecule complex is determined and correlated to the presence or amount of the analyte contacted with the analyte-binding molecule prior to (c).

6. The method of claim 5, wherein the change in conductance is inversely proportional to the amount of the analyte.

7. The method of claim 4, wherein the analyte-binding molecule is conjugated to a signal enhancer.

8. The method of claim 7, wherein the signal enhancer is selected from the group consisting of a metal nanoparticle, a quantum dot, a carbon-based nanomaterial, a silicon particle, a silica particle, an organic molecule, and a mixture thereof.

9. The method of claim 8, wherein the metal nanoparticle consists of a metal selected from the group consisting of copper, gold, silver and platinum.

10. The method of claim 1, wherein the semiconducting nanostructure comprises a nanostructure selected from the group consisting of a nanotube, a nanowire, a nanopillar, a nanorod, a nanosphere, and a mixture thereof.

11. The method of claim 10, wherein the semiconducting nanostructure comprises a carbon semiconductor nanotube or nanowire.

12. The method of claim 11, wherein the carbon semiconductor nanotube or nanowire is selected from the group consisting of a single nanotube or nanowire, multiple nanotubes or nanowires, or a network of nanotubes or nanowires.

13. The method of claim 1, wherein the semiconducting nanostructure is deposited across metal electrodes.

14. The method of claim 12, wherein the carbon semiconductor nanotube or nanowire is a network of nanotubes or nanowires in the form of a flexible, laminated network.

15. The method of claim 1, wherein the nanostructure is placed in a microfluidic channel.

16. The method of claim 15, wherein the nanostructure in the microfluidic channel forms part of a transistor or a resistor.

17. The method of claim 16, wherein the transistor is a field effect transistor (FET).

18. The method of claim 17, wherein the transistor is a liquid-gated field effect transistor (LGFET).

19. The method of claim 1, wherein each carrier molecule is coupled to 2 or more analyte molecules.

20. The method of claim 1, wherein the coupling of the analyte to the carrier molecule is covalent coupling.

21. The method of claim 1, wherein the coupling of the analyte-binding molecule to the nanostructure, or the immobilization of the analyte:carrier molecule complex on the nanostructure, is covalent.

22. The method of claim 1, wherein the analyte has a size of below 1 kD or below 500 D.

23. The method of claim 1, wherein the analyte is a small organic molecule or immunological hapten.

24. The method of claim 23, wherein the analyte is selected from the group consisting of a drug, toxin, pesticide and metabolites thereof.

25. The method of claim 24, wherein the drug is morphine, or a derivative, or metabolite thereof.

26. The method of claim 24, wherein the pesticide is atrazine or 2,4-dichlorophenoxyacetic acid.

27. The method of claim 1, wherein the carrier molecule has a size of above 1 kDa or above 5 kDa.

28. The method of claim 27, wherein the carrier molecule is an albumin.

29. The method of claim 28, wherein the carrier molecule is bovine serum albumin.

30. The method of claim 1, wherein the analyte-binding molecule specifically binds the analyte.

31. The method of claim 1, wherein the analyte-binding molecule is selected from the group consisting of an antibody, antibody fragment, antibody variant, antibody-like molecule, or receptor protein.

32. A fluidic sensor device for determining the presence of an analyte in a fluid sample, the sensor device comprising:
a substrate comprising a microchannel, wherein the microchannel comprises a detection area, and wherein the detection area is arranged to be contactable by the fluid sample flowing through the microchannel, wherein the detection area comprises a network of semiconducting nanostructures, wherein an analyte:carrier molecule complex or an analyte-binding molecule is coupled to the network of nanostructures; and
a first electrode and a second electrode, wherein the first electrode and the second electrode are electrically connected to the detection area.

33. The fluidic sensor device of claim 32, wherein the sensor device is a transistor or resistor.

34. The fluidic sensor device of claim 33, wherein the sensor device is a field effect transistor (FET).

35. The fluidic sensor device of claim 34, wherein the sensor device is a liquid-gated field effect transistor (LGFET).
